(19) United States
(12) Patent Application Publication
(10) Pub. No.: US 2018/0132768 A1
(43) Pub. Date: May 17, 2018

(54) LIVING BODY MONITORING SYSTEM, PORTABLE ELECTRONIC APPARATUS, LIVING BODY MONITORING PROGRAM, COMPUTER READABLE RECORDING MEDIUM, LIVING BODY MONITORING METHOD, DISPLAY DEVICE AND DISPLAY METHOD

(71) Applicant: SEIKO EPSON CORPORATION, Tokyo (JP)

(72) Inventors: Hideo SASAHARA, Chino-shi (JP); Kyohei ISOHATA, Matsumoto-shi (JP)

(73) Assignee: SEIKO EPSON CORPORATION, Tokyo (JP)

(21) Appl. No.: 15/812,490

(22) Filed: Nov. 14, 2017

(30) Foreign Application Priority Data
Nov. 16, 2016 (JP) 2016-223387

(51) Int. Cl. A61B 5/145; A61B 5/00; A61B 5/22; A61B 5/024

(52) U.S. Cl. CPC A61B 5/14542; A61B 5/746; A61B 5/222; A61B 5/7455; A61B 5/7405; A61B 5/02444; A61B 5/743

(57) ABSTRACT

A living body monitoring system includes an output unit that outputs information regarding a load applied on a user's body on the basis of information regarding a change in an altitude of a point where the user is located and information regarding a change in a blood oxygen amount of the user.

### BACKGROUND

### 1. Technical Field

The present invention relates to a living body monitoring system, a portable electronic apparatus, a living body monitoring program, a computer readable recording medium, living body monitoring method, a display device and a display method.

### 2. Related Art

Oxygen concentration in the atmosphere is low (oxygen partial pressure is low) at high places, and thus there is a case where a person suffers from altitude sickness symptoms due to lack of experience or self-confidence in activities at high places such as mountain climbing. Due to the increasing number of behavior disasters and distress caused by altitude sickness symptoms, there are great demands for methods for safely performing activities at high places. For example, JP-A-2013-34767 discloses an apparatus which determines a degree of danger (altitude sickness) on a user during mountain climbing on the basis of altitude information.

However, there is a problem in that a load applied on a user's body is determined on the basis of only altitude (elevation) of a point where the user is located, and this may be insufficient in terms of accuracy.

### SUMMARY

An advantage of some aspects of the invention is to provide a living body monitoring system, a portable electronic apparatus, a living body monitoring program, a computer readable recording medium, a living body monitoring method, a display device and a display method, capable of acquiring information regarding a load applied on a user's body caused by altitude with high accuracy.

The invention can be implemented as the following forms or application examples.

### Application Example 1

A living body monitoring system according to this application example includes an output unit that outputs information regarding a load applied on a user's body on the basis of information regarding a change in an altitude of a point where the user is located and information regarding a change in a blood oxygen amount of the user.

The information is based on both of a change in an altitude and a blood oxygen amount of a user, and is thus considered to accurately reflect a load on the user's body, for example, in a case where an exercise is continuously performed in this state. Therefore, the user can use the information as a reference for determining whether or not the content of the exercise performed by the user is to be reconsidered.

### Application Example 2

The living body monitoring system according to the application example may further include a storage section that stores information regarding a correspondence relationship between a combination of the information regarding a change in the altitude and the information regarding a change in the blood oxygen amount, and the information regarding the load.

Therefore, according to this application example, it is possible to reduce a calculation amount when acquiring information regarding a load on the user's body on the basis of information regarding a change in an altitude and information regarding a change in a blood oxygen amount.

### Application Example 3

In the living body monitoring system according to the application example, the storage section may store the information regarding the correspondence relationship for each level of physical ability of the user.

Therefore, according to this application example, it is possible to acquire information suitable for each of various users having different levels of physical abilities.

### Application Example 4

In the living body monitoring system according to the application example, the storage section may store the information regarding the correspondence relationship for each sex of the user.

Therefore, according to this application example, it is possible to acquire information suitable for each of various users having different sexes.

### Application Example 5

In the living body monitoring system according to the application example, the storage section may store the information regarding the correspondence relationship according to the age of the user.

Therefore, according to this application example, it is possible to acquire information suitable for each of various users according to the age thereof.

### Application Example 6

In the living body monitoring system according to the application example, the information regarding the correspondence relationship may be set according to a relationship between the information regarding a change in the altitude and the information regarding a change in the blood oxygen amount during normal times.

Therefore, according to this application example, it is possible to cope with variations among individual users.

### Application Example 7

In the living body monitoring system according to the application example, the output unit may output transition in the load along with transition in the blood oxygen amount.

Therefore, a user can check transition in a blood oxygen amount along with transition in a load.

### Application Example 8

In the living body monitoring system according to the application example, the output unit may display the transition in the blood oxygen amount in a graph.

Therefore, a user can visually check transition in a blood oxygen amount along with transition in a load.

### Application Example 9

In the living body monitoring system according to the application example, the output unit may represent the transition in the load by using at least one of a color and a pattern of the graph.

Therefore, a user can intuitively recognize transition in a load on the basis of at least one of a color and a pattern.

### Application Example 10

In the living body monitoring system according to the application example, the output unit may display at least one of a graph of transition in the altitude and a graph of transition in atmospheric pressure along with the graph of transition in the blood oxygen amount.

Therefore, a user can check at least one of a graph of transition in an altitude and a graph of transition in atmospheric pressure along with a graph of transition in a blood oxygen amount.

### Application Example 11

In the living body monitoring system according to the application example, the information regarding the load may include advice to the user.

Therefore, a user can check information regarding a load on the user as advice to the user.

### Application Example 12

In the living body monitoring system according to the application example, the advice may include at least one of advice related to the necessity of rest and advice related to the necessity of doctor's examination.

Therefore, it is possible to recommend doctor's examination to a user at right timing corresponding to a load on the user's body.

### Application Example 13

In the living body monitoring system according to the application example, the information regarding the load may include at least one of information regarding a point where a rest is possible and information regarding a point where a doctor's examination is possible.

Therefore, it is possible to notify a user of at least one of information regarding a point where a rest is possible and information regarding a point where a doctor' s examination is possible at right timing corresponding to a load on the user's body.

### Application Example 14

In the living body monitoring system according to the application example, the information regarding the load may be information which can be recognized by the user through at least one of an auditory sense, a visual sense, and a tactile sense.

Therefore, the information can be recognized by a user through at least one of the user's auditory sense, visual sense, and tactile sense.

### Application Example 15

In the living body monitoring system according to the application example, the information regarding the load may be displayed by using at least one of a color, text, a symbol, a scale, and a pattern which can be recognized through a visual sense by the user.

Therefore, the information can be recognized by a user by using at least one of a color, text, a symbol, a scale, and a pattern.

### Application Example 16

The living body monitoring system according to the application example may further include a portable electronic apparatus which can be carried by the user, and a portable electronic apparatus which can be carried by another user who is different from the user, and at least one of output destinations of the information regarding the load may be the portable electronic apparatus of another user.

Therefore, since another user can understand a load on the user, for example, in a case where the user loses consciousness due to a high load, another user can be aware thereof so as to perform immediate handling.

### Application Example 17

A portable electronic apparatus according to this application example includes an output unit that outputs information regarding a load applied on a user's body on the basis of information regarding a change in an altitude of a point where the user is located and information regarding a change in a blood oxygen amount of the user.

The information is based on both of a change in an altitude and a blood oxygen amount of a user, and is thus considered to accurately reflect a load on the user's body, for example, in a case where an exercise is continuously performed in this state. Therefore, the user can use the information as a reference for determining whether or not the content of the exercise performed by the user is to be reconsidered.

### Application Example 18

A living body monitoring program according to this application example causes a computer to execute outputting information regarding a load applied on a user's body on the basis of information regarding a change in an altitude of a point where the user is located and information regarding a change in a blood oxygen amount of the user.

The information is based on both of a change in an altitude and a blood oxygen amount of a user, and is thus considered to accurately reflect a load on the user's body, for example, in a case where an exercise is continuously performed in this state. Therefore, the user can use the information as a reference for determining whether or not the content of the exercise performed by the user is to be reconsidered.

### Application Example 19

A computer readable recording medium according to this application example records a program causing a computer to execute outputting information regarding a load applied on a user's body on the basis of information regarding a change in an altitude of a point where the user is located and information regarding a change in a blood oxygen amount of the user.

The information is based on both of a change in an altitude and a blood oxygen amount of a user, and is thus considered to accurately reflect a load on the user's body, for example, in a case where an exercise is continuously performed in this state. Therefore, the user can use the information as a reference for determining whether or not the content of the exercise performed by the user is to be reconsidered.

### Application Example 20

A living body monitoring method according to this application example includes outputting information regarding a load applied on a user's body on the basis of information regarding a change in an altitude of a point where the user is located and information regarding a change in a blood oxygen amount of the user.

The information is based on both of a change in an altitude and a blood oxygen amount of a user, and is thus considered to accurately reflect a load on the user's body, for example, in a case where an exercise is continuously performed in this state. Therefore, the user can use the information as a reference for determining whether or not the content of the exercise performed by the user is to be reconsidered.

### Application Example 21

A portable electronic apparatus according to this application example outputs information regarding a load applied on a user's body on the basis of information regarding a change in an altitude of a point where the user is located and information regarding a change in a blood oxygen amount of the user.

The information is based on both of a change in an altitude and a blood oxygen amount of a user, and is thus considered to accurately reflect a load on the user's body, for example, in a case where an exercise is continuously performed in this state. Therefore, the user can use the information as a reference for determining whether or not the content of the exercise performed by the user is to be reconsidered.

### Application Example 22

A display device according to this application example displays a load applied on a user's body on the basis of information regarding a change in an altitude of a point where the user is located and information regarding a change in a blood oxygen amount of the user, along with the blood oxygen amount.

Therefore, the user can check a blood oxygen amount of the user along with a load applied on the user's body.

### Application Example 23

The display device according to the application example may display transition in the load along with transition in the blood oxygen amount.

Therefore, a user can check transition in a blood oxygen amount along with transition in a load.

### Application Example 24

The display device according to the application example may display the transition in the blood oxygen amount in a graph.

Therefore, a user can visually check transition in a blood oxygen amount along with transition in a load.

### Application Example 25

The display device according to the application example may represent the transition in the load by using at least one of a color and a pattern of the graph.

Therefore, a user can intuitively recognize transition in a load on the basis of at least one of a color and a pattern.

### Application Example 26

The display device according to the application example may display at least one of a graph of transition in the altitude and a graph of transition in atmospheric pressure along with the graph of transition in the blood oxygen amount.

Therefore, a user can check at least one of a graph of transition in an altitude and a graph of transition in atmospheric pressure along with a graph of transition in a blood oxygen amount.

### BRIEF DESCRIPTION OF THE DRAWINGS

The invention will be described with reference to the accompanying drawings, wherein like numbers reference like elements.

FIG. 1 illustrates an example for explaining an overview of a system.

FIG. 2 illustrates an example of a functional block diagram of a sensor device and an electronic apparatus.

FIG. 3 illustrates an example of a functional block diagram of an information terminal and a server.

FIG. 4 illustrates an example of mounting the sensor device and the electronic apparatus.

FIG. 5 illustrates an example of an exterior of the sensor device.

FIG. 6 illustrates another example of an exterior of the sensor device.

FIG. 7 illustrates an example of a table for normal people.

FIG. 8 illustrates an example of a table for intermediates.

FIG. 9 illustrates an example of a table for athletes.

FIG. 10 illustrates an example of a table for men.

FIG. 11 illustrates an example of a table for women.

FIG. 12 illustrates an example of a table for adults.

FIG. 13 illustrates an example of a table for children.

FIG. 14 illustrates an example of a table for old people.

FIG. 15 illustrates an example of a screen displaying status information of a user.

FIG. 16 illustrates another example of a screen displaying status information of a user.

FIG. 17 illustrates still another example of a screen displaying status information of a user.

FIG. 18 illustrates still another example of a screen displaying status information of a user.

FIG. 19 illustrates still another example of a screen displaying status information of a user.

FIG. 20 illustrates an example of a flowchart for explaining a process performed by a processing section.

FIG. 21 illustrates an example of a head mounted display.

### DESCRIPTION OF EXEMPLARY EMBODIMENTS

Hereinafter, a preferred embodiment of the invention will be described with reference to the drawings. The embodiments described below are not intended to improperly limit the content of the invention disclosed in the appended claims. In addition, all constituent elements described below are not essential constituent elements of the invention.

### 1. SYSTEM

### 1-1. Overview of System

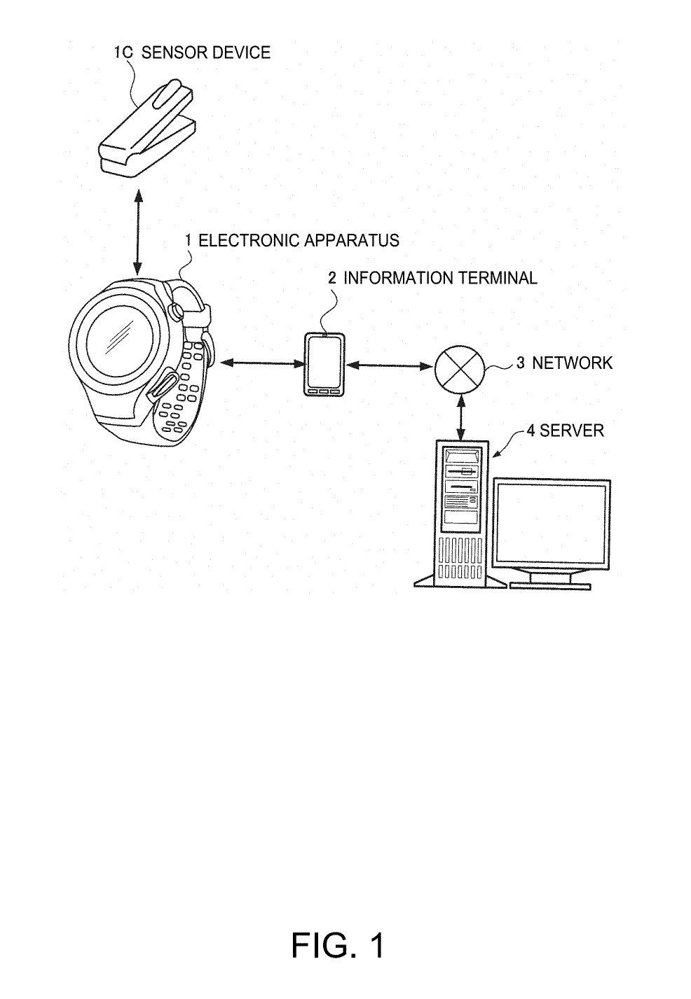

As illustrated in FIG. 1, a system (an example of a living body monitoring system) of the present embodiment includes an electronic apparatus 1 (an example of a portable electronic apparatus), a sensor device 1C, an information terminal 2, and a server 4. Each of the information terminal 2 and the server 4 is connectable to a network 3 such as the Internet, and the information terminal 2 and the server 4 can perform communication with each other via the network 3. The electronic apparatus 1 can perform communication with the information terminal 2 via short-range radio communication or the like. The sensor device 1C can perform communication with the electronic apparatus 1 via short-range radio communication or the like. The sensor device 1C and the information terminal 2 may directly communicate with each other via short-range radio communication or the like (a user may use the electronic apparatus 1 as a master device, and may use the sensor device 1C as a slave device). In the system of the present embodiment, some of the electronic apparatus 1, the sensor device 1C, the information terminal 2, and the server 4 may be omitted.

Each of the electronic apparatus 1 and the sensor device 1C is, for example, a waterproof type apparatus on which various sensors are mounted, and acquires measured data (in which output data from various sensors are associated with time points) during life or exercise by driving at least one sensor. At least one of the electronic apparatus 1 and the sensor device 1C is mounted a user's body during the life or exercise. The “exercise” is assumed to include an exercise in which a user moves along a rolling route. Hereinafter, an exercise (mountain climbing) of a user moving along a mountain climbing route is assumed.

A mounting location of the electronic apparatus 1 is, for example, a part (forearm) from the elbow to the hand. The electronic apparatus 1 is formed of a wrist type (wristwatch type) electronic apparatus (outdoor watch) so that measured data regarding a user's living body is acquired in a contact or noncontact manner, or the user can view the electronic apparatus 1 when necessary, and a mounting location of the electronic apparatus 1 is the wrist of the user. A mounting tool (belt) suitable for a shape of the wrist is used when the electronic apparatus 1 is mounted on the wrist. The mounting tool is provided with fitting holes and a buckle. The buckle is formed of a buckle frame and an engagement portion (protrusion rod). A plurality of fitting holes are provided in the mounting tool, and the engagement portion of the buckle is inserted into any one of the plurality of fitting holes so that the electronic apparatus 1 is mounted on the user. The plurality of fitting holes are provided along a longitudinal direction of the mounting tool.

A mounting location of the sensor device 1C may be selected as appropriate by the user according to the purpose of exercise, the purpose of measurement, or the like. A mounting location of the sensor device 1C is, for example, any one of the user's fingertip, head, upper arm, forearm, waist, chest, thigh, crus, and ankle. The example illustrated in FIG. 1 shows the sensor device 1C of a type of being mounted on the fingertip. When the sensor device 1C is mounted on a part of the body, a mounting tool (a belt or a clip) suitable for a shape of the part or a shape of an exercise wear may be used.

The information terminal 2 is an information terminal used by the user of the electronic apparatus 1 or the sensor device 1C, and is formed of, for example, a smart phone, a portable or desktop personal computer (PC) or a tablet PC. The information terminal 2 is used for the user to perform settings on the electronic apparatus 1 or the sensor device 1C, for example, before using the electronic apparatus 1 or the sensor device 1C. The information terminal 2 is used for the user to read measured data regarding the user from the electronic apparatus 1 or the sensor device 1C, for example, after using the electronic apparatus 1 or the sensor device 1C, or to upload the read measured data to the server 4. A user of the information terminal 2 may be manager observing the life of a user wearing the electronic apparatus 1 or the sensor device 1C, or may be a coach coaching an exercise of a user wearing the electronic apparatus 1 or the sensor device 1C.

The server 4 provides information related to use of the electronic apparatus 1 to a user of the electronic apparatus 1 or the sensor device 1C, or manages measured data acquired by the electronic apparatus 1 or the sensor device 1C for each user. The information provided by the server 4 may include at least one of a program for operating the information terminal 2, a program for operating the electronic apparatus 1, and a program for operating the sensor device 1C. The program of each apparatus may be downloaded to each apparatus via the network 3, and may be downloaded to each apparatus via a recording medium. This is also the same for a program of the server 4.

### 1-2. Configuration of System

### 1-2-1. Configuration of Sensor Device

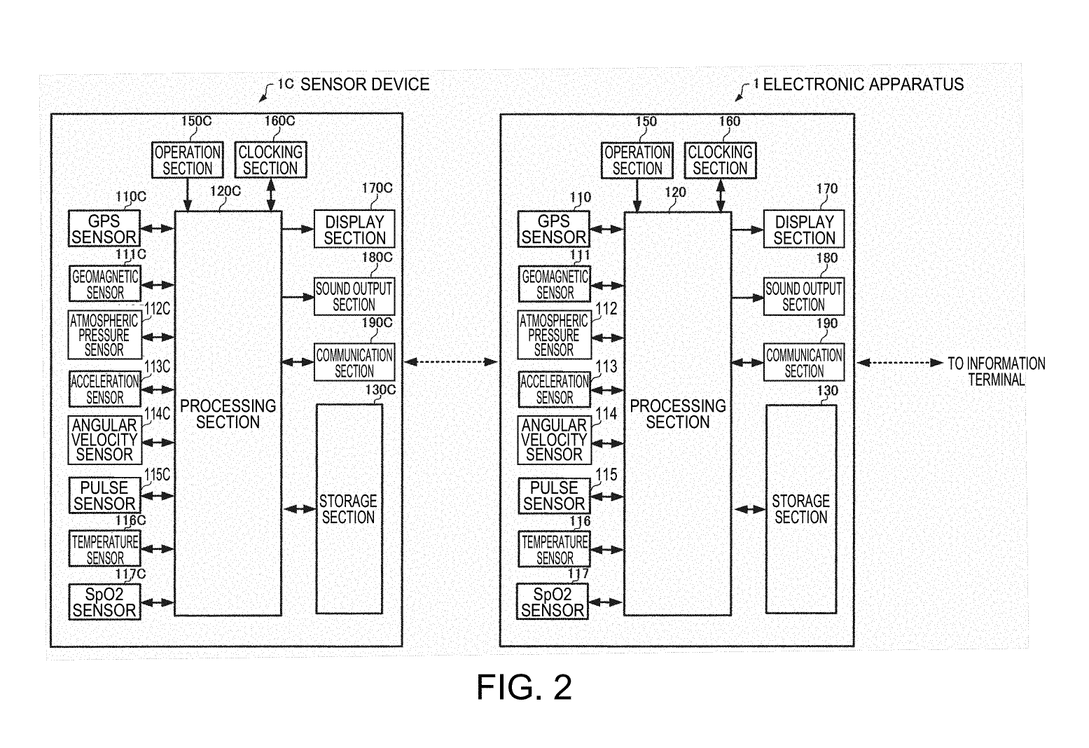

As illustrated in FIG. 2, the sensor device 1C is configured to include a GPS sensor 110C, a geomagnetic sensor 111C, an atmospheric pressure sensor 112C, an acceleration sensor 113C, an angular velocity sensor 114C, a pulse sensor 115C, a temperature sensor 116C, an SpO₂ sensor 117C, a processing section 120C, a storage section 130C, an operation section 150C, a clocking section 160C, a display section 170C, a sound output section 180C, a communication section 190C, and the like. However, a configuration of the sensor device 1C may have a configuration in which some of the constituent elements are deleted or changed, or may have a configuration in which other constituent elements (for example, a humidity sensor and an ultraviolet sensor) are added thereto.

The GPS sensor 110C is a sensor which generates positioning data (data such as latitude, longitude, altitude, and speed vector) indicating a position and the like of the sensor device 1C and outputs the data to the processing section 120C, and is formed of, for example, a global positioning system (GPS) receiver. The GPS sensor 110C receives an electric wave with a satellite signal of a predetermined frequency bandwidth incoming from the outside by using a GPS antenna (not illustrated), extracts a GPS signal sent from a GPS satellite therefrom, and also generates positioning data indicating a position or the like of the sensor device 1C on the basis of the GPS signal.

The geomagnetic sensor 111C is a sensor which detects a geomagnetic vector indicating a direction of a magnetic field of the earth, viewed from the sensor device 1C, and generates, for example, geomagnetic data indicating magnetic flux densities in three-axis directions which are orthogonal to each other. For example, a magnet resistive (MR) element, a magnet impedance (MI) element, or a hole element is used for the geomagnetic sensor 111C.

The atmospheric pressure sensor 112C is a sensor which detects an ambient atmospheric pressure (atmospheric pressure), and includes, for example, a pressure sensitive element of a type (vibration type) using a change in a resonance frequency of a vibrator element. The pressure sensitive element is a piezoelectric vibrator made of a piezoelectric material such as quartz crystal, Lithium Niobate, or Lithium Tantalate, and employs, for example, a tuning fork type vibrator, a dual-tuning fork type vibrator, an AT vibrator (thickness shear vibrator), or a surface acoustic wave (SAW) resonator. An output from the atmospheric pressure sensor 112C may be used to correct positioning data. The atmospheric pressure sensor 112C may be a MEMS type atmospheric pressure sensor manufactured by using, for example, a semiconductor manufacturing technique. Specifically, the atmospheric pressure sensor 112C includes a diaphragm portion which is bent and deformed by received pressure, and a distortion detection element which detects bending of the diaphragm portion. The diaphragm portion is made of, for example, silicon. The distortion detection element is, for example, a piezoelectric resistive element.

The acceleration sensor 113C is an inertial sensor which detects respective accelerations in the three-axis directions which intersect (are ideally orthogonal to) each other, and outputs digital signals (acceleration data) corresponding to magnitudes and directions of the detected three-axis accelerations. The outputs from the acceleration sensor 113C may be used to correct position information included in the positioning data from the GPS sensor 110C.

The angular velocity sensor 114C is an inertial sensor which detects respective angular velocities in the three-axis directions which intersect (are ideally orthogonal to) each other, and outputs digital signals (angular velocity data) corresponding to magnitudes and directions of the measured three-axis angular velocities. The outputs from the angular velocity sensor 114C may be used to correct position information included in the positioning data from the GPS sensor 110C.

The pulse sensor 115C is a sensor which generates a signal indicating a pulse of the user and outputs the signal to the processing section 120C, and includes, for example, a light source such as an LED light source which applies measurement light having an appropriate wavelength toward a blood vessel under the skin, and a light receiving element which detects a change in the intensity of light generated at the blood vessel according to the measurement light. The “pulse” is a pulsation generated when blood is pushed out due to beating of the heart, and thus a change in the arterial pressure is transmitted to the artery of the whole body, and a pulsation per minute is called a “pulse rate”. A “pulse” appearing in a blood vessel of a part separated from the heart has high correlation with beating of the heart, that is, “heartbeat”, and, thus, herein, the “pulse” is used in the same meaning as the “heartbeat”.

The temperature sensor 116C is a deep body temperature sensor which detects a deep body temperature of a user's body. The temperature sensor 116C includes, for example, a base material, a first temperature sensor which measures the temperature of a first position in the base material as a first temperature, a second temperature sensor which measures the temperature of a second position which is different from the first position in the base material as a second temperature, a temperature sensor which measures the temperature of an outer shell body surrounding the base material as a medium temperature of the outer shell body inside, and a calculation portion which calculates a deep body temperature of a subject by using the first temperature, the second temperature, and the medium temperature. The temperature sensor 116C calculates a constant which correlates the medium temperature, the first temperature, and the second temperature with each other on the basis of the first temperature (Tb1) and the second temperature (Tp1) when the medium temperature is a first medium temperature (Tout1), the first temperature (Tb2) and the second temperature (Tp2) when the medium temperature is a second medium temperature (Tout2), and the first temperature (Tb3) and the second temperature (Tp3) when the medium temperature is a third medium temperature (Tout3), and calculates the deep body temperature (Tc). Each of such temperature sensors is, for example, a temperature sensitive element which outputs a signal (for example, a voltage corresponding to a temperature) corresponding to an ambient temperature. The temperature sensor may output a digital signal corresponding to a temperature. The temperature sensor has a pressure sensitive element of a type (vibration type) using a change in a resonance frequency of a vibrating element corresponding to an ambient temperature. The pressure sensitive element is a piezoelectric vibrator made of a piezoelectric material such as quartz crystal, Lithium Niobate, or Lithium Tantalate, and employs, for example, a tuning fork type vibrator, a dual-tuning fork type vibrator, an AT vibrator (thickness shear vibrator), or a SAW resonator. Alternatively, the temperature sensor may be formed of a temperature sensitive element which detects a temperature with a thermocouple or a thermistor.

The SpO₂ sensor 117C is a sensor which outputs a signal related to arterial blood oxygen saturation (hereinafter, referred to as “SpO₂”; an example of a blood oxygen amount) of the user. The SpO₂ sensor 117C includes an irradiation unit that irradiates the inside of a blood vessel under the skin of the user with light having a predetermined wavelength, and a light reception unit which receives reflected light returning from the blood vessel of the user so as to output a signal related to SpO₂ of the user. Generally, oxygenated hemoglobin HbO₂ and deoxygenated hemoglobin Hb have different light absorption spectra, and, in a case where light having a relative long wavelength λ1 (>λ) is applied, a light absorption coefficient at the light is greater in the oxygenated hemoglobin HbO₂, and thus the intensity (an output value V1 in the light reception unit) of transmitted light or reflected light at the light is an index value indicating an amount of oxygenated hemoglobin in a blood vessel. Similarly, in a case where light having a relative long wavelength λ2 (<λ) is applied, a light absorption coefficient at the light is greater in the deoxygenated hemoglobin Hb, and thus the intensity (an output value V2 in the light reception unit) of transmitted light or reflected light at the light is an index value indicating an amount of deoxygenated hemoglobin in a blood vessel. Thus, V1/(V1+V2) is an index value indicating a proportion of oxygenated hemoglobin, that is, a value having a correlation with the blood oxygen saturation SpO₂. As mentioned above, SpO₂ can be obtained on the basis of transmitted light and reflected light at light having two different wavelengths by using the characteristic that the oxygenated hemoglobin HbO₂ and the deoxygenated hemoglobin Hb have different light absorption spectra. In the above description, a simple method has been described, but there are various modifications of a method of obtaining SpO₂ or information similar to SpO₂ by using infrared light red light, and the SpO₂ sensor 117C may widely employ the modifications. The absorbance of the oxygenated hemoglobin HbO₂ and the deoxygenated hemoglobin Hb at one wavelength is preferably clearly different from the absorbance at the other wavelength, and a wavelength to be used is not limited to wavelengths of infrared light and red light. For example, there may be a modification in which one of infrared light and red light is changed to green light. In the following description, a description will be made of a case where the processing section 120C calculates a value of SpO₂ (unit: %) on the basis of a signal output from the SpO₂ sensor 117C, but a format of SpO₂ may be variously modified.

The storage section 130C is formed of, for example, various integrated circuit (IC) memories such as a read only memory (ROM), a flash ROM, and a random access memory (RAM), or a computer readable recording medium such as a hard disk or a memory card. The storage section 130C is formed of, for example, one or a plurality of IC memories, and includes a ROM storing data such as a program, and a RAM serving as a work region of the processing section 120C. The RAM may include a nonvolatile RAM.

The operation section 150C is formed of, for example, a button, a key, a microphone, and a touch panel so as to have a voice recognition function (using the microphone (not illustrated)) and an action detection function (using the acceleration sensor 113C or the like), and performs a process of converting an instruction from a user into an appropriate signal which is then sent to the processing section 120C.

The clocking section 160C is formed of, for example, a real time clock (RTC) IC or the like, and generates time data such as year, month, day, hour, minute, and second, and sends the data to the processing section 120C.

The display section 170C is formed of, for example, a liquid crystal display (LCD), an organic electroluminescence (EL) display, an electrophoretic display (EPD), or a touch panel display, and displays various images in response to instructions from the processing section 120C.

The sound output section 180C is formed of, for example, a speaker, a buzzer, or a vibrator, and generates various sounds (or vibration) in response to instructions from the processing section 120C.

The communication section 190C performs a variety of control for establishing data communication between the sensor device 1C and the electronic apparatus 1 (or the information terminal 2). The communication section 190C is configured to include a transceiver based on a short-range wireless communication standard such as Bluetooth (registered trademark) (including Bluetooth Low Energy (BTLE)), Wi-Fi (registered trademark) (Wireless Fidelity), Zigbee (registered trademark), Near field communication (NFC), or ANT+ (registered trademark).

The processing section 120C is formed of, for example, a micro processing unit (MPU), a digital signal processor (DSP), and an application specific integrated circuit (ASIC). The processing section 120C performs various processes according to a program stored in the storage section 130C, and various commands which are input by the user via the operation section 150C. The processes in the processing section 120C include, for example, data processing on data which is generated by the GPS sensor 110C, the geomagnetic sensor 111C, the atmospheric pressure sensor 112C, the acceleration sensor 113C, the angular velocity sensor 114C, the pulse sensor 115C, the temperature sensor 116C, the SpO₂ sensor 117C, the clocking section 160C, and the like, display processing for displaying an image on the display section 170C, sound output processing for outputting sounds from the sound output section 180C. The processing section 120C performs a process of receiving a control command from the electronic apparatus 1 via the communication section 190C, or various computation processes on data which is received from the electronic apparatus 1 via the communication section 190C, according to various programs. The processing section 120C performs a process of reading data from the storage section 130C, and transmitting the data to the electronic apparatus 1 in a predetermined format via the communication section 190C, according to various programs. The processing section 120C performs a process of transmitting various pieces of information to the electronic apparatus 1 via the communication section 190C, and displaying various screens on the basis of information received from the electronic apparatus 1, according to various programs. The processing section 120C performs other various control processes. For example, the processing section 120C performs a process of displaying images (images, moving images, text, symbols, and the like) on the display section (display) 170C on the basis of at least some of the information received by the communication section 190C, and the information stored in the storage section 130C. A vibration mechanism may be provided in the sensor device 1C, and various pieces of information may be converted into vibration information by the vibration mechanism so as to be presented to the user.

### 1-2-2. Configuration of Electronic Apparatus

As illustrated in FIG. 2, the electronic apparatus 1 is configured to include a GPS sensor 110, a geomagnetic sensor 111, an atmospheric pressure sensor 112, an acceleration sensor 113, an angular velocity sensor 114, a pulse sensor 115, a temperature sensor 116, a blood oxygen saturation sensor (hereinafter, referred to as “SpO₂ sensor”) 117, a processing section 120, a storage section 130, an operation section 150, a clocking section 160, a display section 170, a sound output section 180, a communication section 190, and the like. However, the electronic apparatus 1 may have a configuration in which some of the constituent elements are deleted or changed, or may have a configuration in which other constituent elements (for example, a humidity sensor and an ultraviolet sensor) are added thereto.

The GPS sensor 110 is a sensor which generates positioning data (data such as latitude, longitude, altitude, and speed) indicating a position and the like of the electronic apparatus 1 and outputs the data to the processing section 120, and is formed of, for example, a global positioning system (GPS) receiver. The GPS sensor 110 receives an electric wave with a satellite signal of a predetermined frequency bandwidth incoming from the outside by using a GPS antenna (not illustrated), extracts a GPS signal sent from a GPS satellite therefrom, and also generates positioning data indicating a position or the like of the electronic apparatus 1 on the basis of the GPS signal.

The geomagnetic sensor 111 is a sensor which detects a geomagnetic vector indicating a direction of a magnetic field of the earth, viewed from the electronic apparatus 1, and generates, for example, geomagnetic data indicating magnetic flux densities in three-axis directions which are orthogonal to each other. For example, a magnet resistive (MR) element, a magnet impedance (MI) element, or a hole element is used for the geomagnetic sensor 111.

The atmospheric pressure sensor 112 is a sensor which detects an ambient atmospheric pressure (atmospheric pressure), and includes, for example, a pressure sensitive element of a type (vibration type) using a change in a resonance frequency of a vibrator element. The pressure sensitive element is a piezoelectric vibrator made of a piezoelectric material such as quartz crystal, Lithium Niobate, or Lithium Tantalate, and employs, for example, a tuning fork type vibrator, a dual-tuning fork type vibrator, an AT vibrator (thickness shear vibrator), or a surface acoustic wave (SAW) resonator. An output from the atmospheric pressure sensor 112 may be used to correct positioning data. The atmospheric pressure sensor 112 may be a MEMS type atmospheric pressure sensor manufactured by using, for example, a semiconductor manufacturing technique. Specifically, the atmospheric pressure sensor 112 includes a diaphragm portion which is bent and deformed by received pressure, and a distortion detection element which detects bending of the diaphragm portion. The diaphragm portion is made of, for example, silicon. The distortion detection element is, for example, a piezoelectric resistive element.

The acceleration sensor 113 is an inertial sensor which detects respective accelerations in the three-axis directions which intersect (are ideally orthogonal to) each other, and outputs digital signals (acceleration data) corresponding to magnitudes and directions of the detected three-axis accelerations. The outputs from the acceleration sensor 113 may be used to correct position information included in the positioning data from the GPS sensor 110.

The angular velocity sensor 114 is an inertial sensor which detects respective angular velocities in the three-axis directions which intersect (are ideally orthogonal to) each other, and outputs digital signals (angular velocity data) corresponding to magnitudes and directions of the measured three-axis angular velocities. The outputs from the angular velocity sensor 114 may be used to correct position information included in the positioning data from the GPS sensor 110.

The pulse sensor 115 is a sensor which generates a signal indicating a pulse of the user and outputs the signal to the processing section 120, and includes, for example, a light source such as a light emitting diode (LED) light source which applies measurement light having an appropriate wavelength toward a blood vessel under the skin, and a light receiving element which detects a change in the intensity of light generated at the blood vessel according to the measurement light. The “pulse” is a pulsation generated when blood is pushed out due to beating of the heart, and thus a change in the arterial pressure is transmitted to the artery of the whole body, and a pulsation per minute is called a “pulse rate”. A “pulse” appearing in a blood vessel of a part separated from the heart has high correlation with beating of the heart, that is, “heartbeat”, and, thus, herein, the “pulse” is used in the same meaning as the “heartbeat”.

The temperature sensor 116 is a deep body temperature sensor which detects a deep body temperature of a user's body. The temperature sensor 116 includes, for example, a base material, a first temperature sensor which measures the temperature of a first position in the base material as a first temperature, a second temperature sensor which measures the temperature of a second position which is different from the first position in the base material as a second temperature, a temperature sensor which measures the temperature of an outer shell body surrounding the base material as a medium temperature of the outer shell body inside, and a calculation portion which calculates a deep body temperature of a subject by using the first temperature, the second temperature, and the medium temperature. The temperature sensor 116 calculates a constant which correlates the medium temperature, the first temperature, and the second temperature with each other on the basis of the first temperature (Tb1) and the second temperature (Tp1) when the medium temperature is a first medium temperature (Tout1), the first temperature (Tb2) and the second temperature (Tp2) when the medium temperature is a second medium temperature (Tout2), and the first temperature (Tb3) and the second temperature (Tp3) when the medium temperature is a third medium temperature (Tout3), and calculates the deep body temperature (Tc). Each of such temperature sensors is, for example, a temperature sensitive element which outputs a signal (for example, a voltage corresponding to a temperature) corresponding to an ambient temperature. The temperature sensor may output a digital signal corresponding to a temperature. The temperature sensor has a pressure sensitive element of a type (vibration type) using a change in a resonance frequency of a vibrating element corresponding to an ambient temperature. The pressure sensitive element is a piezoelectric vibrator made of a piezoelectric material such as quartz crystal, Lithium Niobate, or Lithium Tantalate, and employs, for example, a tuning fork type vibrator, a dual-tuning fork type vibrator, an AT vibrator (thickness shear vibrator), or a SAW resonator. Alternatively, the temperature sensor may be formed of a temperature sensitive element which detects a temperature with a thermocouple or a thermistor.

The SpO₂ sensor 117 is a sensor which outputs a signal related to arterial blood oxygen saturation (hereinafter, referred to as “SpO₂”; an example of a blood oxygen amount) of the user. The SpO₂ sensor 117 includes an irradiation unit that irradiates the inside of a blood vessel under the skin of the user with light having a predetermined wavelength, and a light reception unit which receives reflected light returning from the blood vessel of the user so as to output a signal related to SpO₂ of the user. Generally, oxygenated hemoglobin HbO₂ and deoxygenated hemoglobin Hb have different light absorption spectra, and, in a case where light having a relative long wavelength λ1 (>λ) is applied, a light absorption coefficient at the light is greater in the oxygenated hemoglobin HbO₂, and thus the intensity (an output value V1 in the light reception unit) of transmitted light or reflected light at the light is an index value indicating an amount of oxygenated hemoglobin in a blood vessel. Similarly, in a case where light having a relative long wavelength λ2 (<λ) is applied, a light absorption coefficient at the light is greater in the deoxygenated hemoglobin Hb, and thus the intensity (an output value V2 in the light reception unit) of transmitted light or reflected light at the light is an index value indicating an amount of deoxygenated hemoglobin in a blood vessel. Thus, V1/(V1+V2) is an index value indicating a proportion of oxygenated hemoglobin, that is, a value having a correlation with the blood oxygen saturation SpO₂. As mentioned above, SpO₂ can be obtained on the basis of transmitted light and reflected light at light having two different wavelengths by using the characteristic that the oxygenated hemoglobin HbO₂ and the deoxygenated hemoglobin Hb have different light absorption spectra. In the above description, a simple method has been described, but there are various modifications of a method of obtaining SpO₂ or information similar to SpO₂ by using infrared light red light, and the SpO₂ sensor 117 may widely employ the modifications. The absorbance of the oxygenated hemoglobin HbO₂ and the deoxygenated hemoglobin Hb at one wavelength is preferably clearly different from the absorbance at the other wavelength, and a wavelength to be used is not limited to wavelengths of infrared light and red light. For example, there may be a modification in which one of infrared light and red light is changed to green light. In the following description, a description will be made of a case where the processing section 120 calculates a value of SpO₂ (unit: %) on the basis of a signal output from the SpO₂ sensor 117, but a format of SpO₂ may be variously modified.

The storage section 130 is formed of, for example, various integrated circuit (IC) memories such as a read only memory (ROM), a flash ROM, and a random access memory (RAM), or a computer readable recording medium such as a hard disk or a memory card. The storage section 130 is formed of, for example, one or a plurality of IC memories, and includes a ROM storing data such as a program, and a RAM serving as a work region of the processing section 120. The RAM may include a nonvolatile RAM.

The operation section 150 is formed of, for example, a button, a key, a microphone, and a touch panel so as to have a voice recognition function (using the microphone (not illustrated)) and an action detection function (using the acceleration sensor 113 or the like), and performs a process of converting an instruction from the user into an appropriate signal which is then sent to the processing section 120.

The clocking section 160 is formed of, for example, a real time clock (RTC) IC or the like, and generates time data such as year, month, day, hour, minute, and second, and sends the data to the processing section 120.

The display section 170 is formed of, for example, a liquid crystal display (LCD), an organic electroluminescence (EL) display, an electrophoretic display (EPD), or a touch panel display, and displays various images in response to instructions from the processing section 120.

The sound output section 180 is formed of, for example, a speaker, a buzzer, or a vibrator, and generates various sounds (or vibration) in response to instructions from the processing section 120.

The communication section 190 performs a variety of control for establishing data communication between the electronic apparatus 1 and the sensor device 1C or the information terminal 2. The communication section 190 is configured to include a transceiver based on a short-range wireless communication standard such as Bluetooth (registered trademark) (including Bluetooth Low Energy (BTLE)), Wi-Fi (registered trademark) (Wireless Fidelity), Zigbee (registered trademark), near field communication (NFC), or ANT+ (registered trademark).

The processing section 120 is formed of, for example, a micro processing unit (MPU), a digital signal processor (DSP), and an application specific integrated circuit (ASIC). The processing section 120 performs various processes according to a program stored in the storage section 130, and various commands which are input by the user via the operation section 150. The processes in the processing section 120 include, for example, data processing on data which is generated by the GPS sensor 110, the geomagnetic sensor 111, the atmospheric pressure sensor 112, the acceleration sensor 113, the angular velocity sensor 114, the pulse sensor 115, the temperature sensor 116, the clocking section 160, and the like, display processing for displaying an image on the display section 170, sound output processing for outputting sounds from the sound output section 180. The processing section 120 performs a process of receiving a control command from the information terminal 2 via the communication section 190, or various computation processes on data which is received from the information terminal 2 via the communication section 190, according to various programs. The processing section 120 performs a process of reading data from the storage section 130, and transmitting the data to the information terminal 2 in a predetermined format via the communication section 190, according to various programs. The processing section 120 performs a process of transmitting various pieces of information to the information terminal 2 via the communication section 190, and displaying various screens on the basis of information received from the information terminal 2, according to various programs. The processing section 120 performs other various control processes. For example, the processing section 120 performs a process of displaying images (images, moving images, text, symbols, and the like) on the display section 170 on the basis of at least some of the information received by the communication section 190, the information received by the communication section 190, and information stored in the storage section 130. A vibration mechanism may be provided in the electronic apparatus 1, and various pieces of information may be converted into vibration information by the vibration mechanism so as to be presented to the user.

### 1-2-3. Configuration of Information Terminal

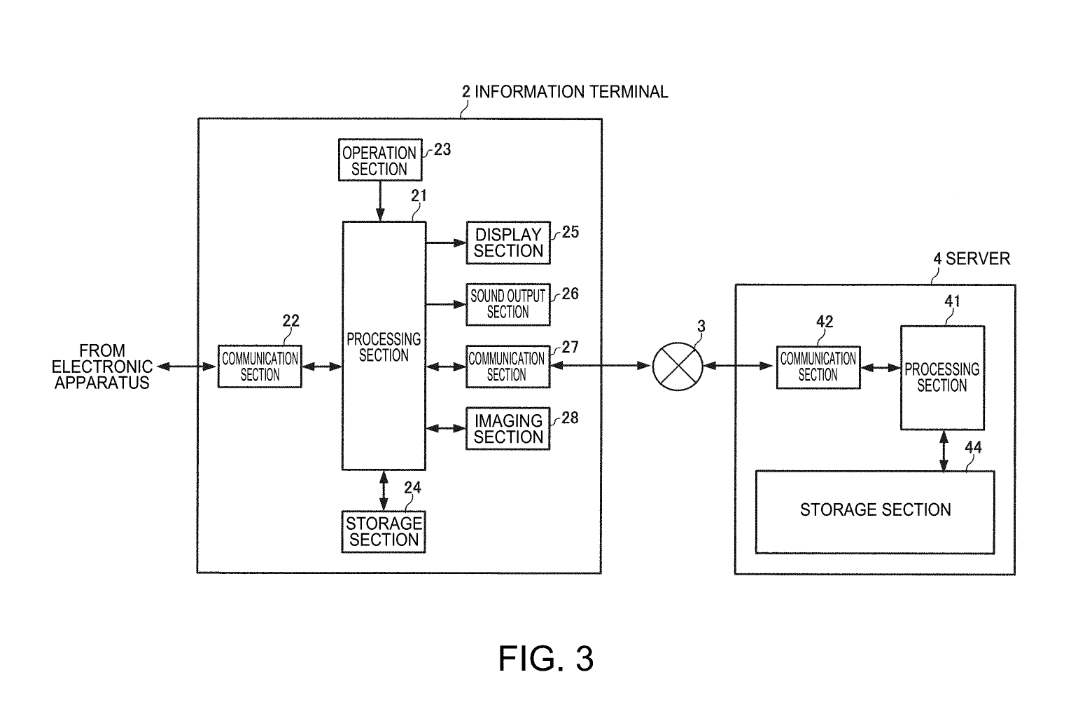

As illustrated in FIG. 3, the information terminal 2 is configured to include a processing section 21, a communication section 22, an operation section 23, a storage section 24, a display section 25, a sound output section 26, a communication section 27, and an imaging section 28. However, the information terminal 2 may have a configuration in which some of the constituent elements are deleted or changed as appropriate, or may have a configuration in which other constituent elements are added thereto.

The communication section 22 performs a process of receiving data (measured data) or the like transmitted from the electronic apparatus 1 (or the sensor device 1C) in a predetermined format and sending the data to the processing section 21, a process of transmitting a control command from the processing section 21 to the electronic apparatus 1 or the sensor device 1C, or the like.

The operation section 23 performs a process of acquiring data corresponding to the user's operation, and sending the data to the processing section 21. The operation section 23 may be, for example, a touch panel display, a button, a key, and a microphone.

The storage section 24 is formed of, for example, various integrated circuit (IC) memories such as a read only memory (ROM), a flash ROM, and a random access memory (RAM), or a computer readable recording medium such as a hard disk or a memory card. The storage section 24 stores programs for the processing section 21 performing various computation processes or control processes, various programs or data for realizing application functions. The storage section 24 is used as a work region of the processing section 21, and temporarily stores data which is acquired from the operation section 23, results of calculation executed by the processing section 21 according to various programs, and the like. The storage section 24 may store data which is required to be preserved for a long period of time among data items generated through processing in the processing section 21.

The display section 25 displays a processing result in the processing section 21 as text, a graph, a table, animation, and other images. The display section 25 may be, for example, a cathode ray tube (CRT), a liquid crystal display (LCD), a touch panel display, and a head mounted display (HMD). A single touch panel display may realize functions of the operation section 23 and the display section 25.

The sound output section 26 outputs a processing result in the processing section 21 as a sound (or vibration) such as a voice or a buzzer sound. The sound output section 26 may be, for example, a speaker or a buzzer.

The communication section 27 performs data communication with a communication section 42 of the server 4 via the network 3. For example, the communication section 27 performs a process of receiving data from the processing section 21 and transmitting the data to the communication section 42 of the server 4 in a predetermined format. For example, the communication section 27 performs a process of receiving information required to display a screen from the communication section 42 of the server 4 and sending the information to the processing section 21, or a process of receiving various pieces of information from the processing section 21 and transmitting the information to the communication section 42 of the server 4.

The imaging section 28 is a camera including a lens, a color imaging element, a focus adjustment mechanism, and the like, and generates a picture of a field generated by the lens as an image with the imaging element. Data (image data) regarding the image acquired by the imaging element is sent to the processing section 21 so as to be preserved in the storage section 24 or displayed on the display section 25.

The processing section 21 (an example of a computer) is formed of, for example, a central processing unit (CPU), a digital signal processor (DSP), and an application specific integrated circuit (ASIC). The processing section 21 performs various processes according to a program stored in the storage section 24, and various commands which are input by the user via the operation section 23. The processes in the processing section 21 include, for example, data processing on data which is generated by the electronic apparatus 1 or the sensor device 1C, display processing for displaying an image on the display section 25, sound output processing for outputting sounds from the sound output section 26, and image processing on an image acquired by the imaging section 28. The processing section 21 may be formed of a single processor, and may be formed of a plurality of processors. The processing section 21 performs a process of transmitting a control command to the electronic apparatus 1 via the communication section 22, or various computation processes on data which is received from the electronic apparatus 1 via the communication section 22, according to various programs. The processing section 21 performs a process of reading data from the storage section 24, and transmitting the data to the server 4 in predetermined format via the communication section 27, according to various programs. The processing section 21 performs a process of transmitting various pieces of information to the server 4 via the communication section 27, and displaying various screens on the basis of information received from the server 4, according to various programs. The processing section 21 performs other various control processes. For example, the processing section 21 performs a process of displaying images (images, moving images, text, symbols, and the like) on the display section 25 on the basis of at least some of the information received by the communication section 27, the information received by the communication section 22, and information stored in the storage section 24. A vibration mechanism may be provided in the information terminal 2, the electronic apparatus 1, or the sensor device 1C, and various pieces of information may be converted into vibration information by the vibration mechanism so as to be presented to the user.

### 1-2-4. Configuration of Server

As illustrated in FIG. 3, the server 4 is configured to include a processing section 41, a communication section 42, and a storage section 44. However, the server 4 may have a configuration in which some of the constituent elements are deleted or changed as appropriate, or may have a configuration in which other constituent elements are added thereto.

The storage section 44 is formed of, for example, various integrated circuit (IC) memories such as a read only memory (ROM), a flash ROM, and a random access memory (RAM), or a computer readable recording medium such as a hard disk or a memory card. The storage section 44 stores a program for the processing section 41 performing various calculation processes or a control process, or various programs or data for realizing application functions. The storage section 44 is used as a work region of the processing section 41, and temporarily stores results of calculation executed by the processing section 41 according to various programs, and the like. The storage section 44 may store data which is required to be preserved for a long period of time among pieces of data generated through processing of the processing section 41.

The communication section 42 performs data communication with the communication section 27 of the information terminal 2 via the network 3. For example, the communication section 42 performs a process of receiving data from the communication section 27 of the information terminal 2, and sending the data to the processing section 41. For example, the communication section 42 performs a process of transmitting information required to display a screen to the communication section 27 of the information terminal 2 in a predetermined format, or a process of receiving information from the communication section 27 of the information terminal 2 and sending the information to the processing section 41.

The processing section 41 performs a process of receiving data from the information terminal 2 via the communication section 42 and storing the data in the storage section 44, according to various programs. The processing section 41 performs a process of receiving various pieces of information from the information terminal 2 via the communication section 42, and transmitting information required to display various screens to the information terminal 2, according to various programs. The processing section 41 performs other various control processes.

### 1-2-5. Variation of System Configuration

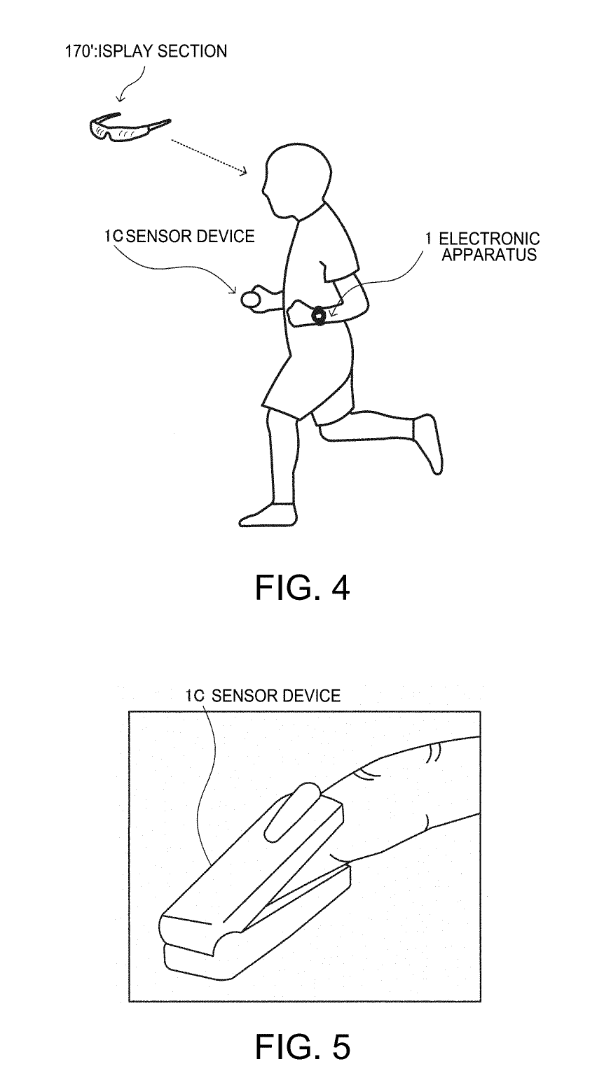

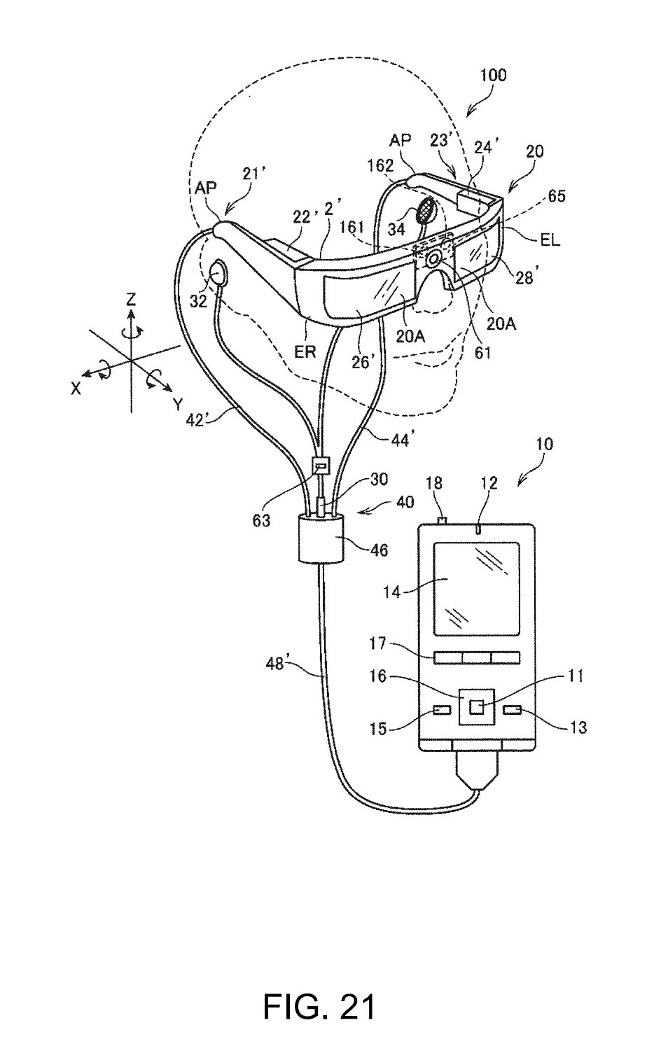

As illustrated in FIG. 4, the user may use a head mounted type display section 170′ called a head mounted display (HMD). The head mounted type display section 170′ performs data communication with the electronic apparatus 1, receives image display data from the electronic apparatus 1, and displays an image in front of the eyes of the user on the basis of the data. As the display section 170′ , for example, a head mounted display (refer to FIG. 21) which will be described later may be used.

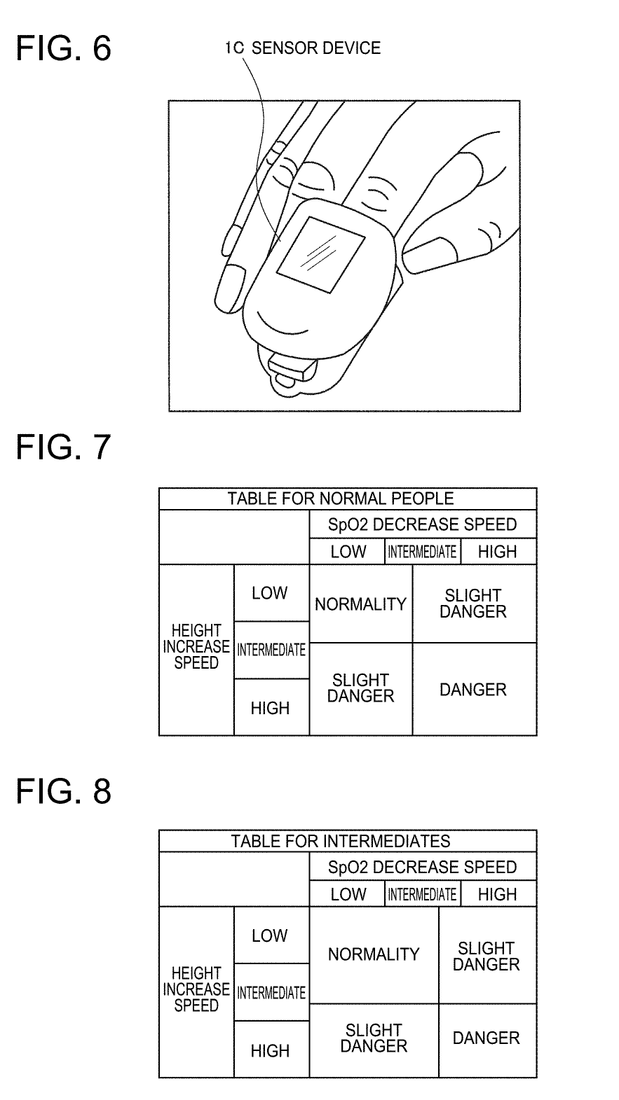

For example, in a case where the user wants SpO₂ to be measured from the fingertip thereof, as illustrated in FIG. 4, the user may mount the clip type sensor device 1C on the fingertip thereof. FIGS. 5 and 6 illustrate examples of exteriors of the clip type sensor device 1C. FIG. 5 illustrates an example of the clip type sensor device 1C from which the display section 170C is omitted, and FIG. 6 illustrates an example of the clip type sensor device 1C into which the display section 170C is integrated.

In a case where the clip type sensor device 1C is used, the user may mount the electronic apparatus 1 on the wrist, and may remotely operate the sensor device 1C via the electronic apparatus 1 by using the electronic apparatus 1 as a master device. For example, the sensor device 1C periodically communicates with the electronic apparatus 1 as a master device, and sequentially transmits measured data acquired by the sensor device 1C to the electronic apparatus 1.

The user may separately mount a plurality of sensor devices 1C on a plurality of parts of the body of the user.

The sensor device 1C may have various forms such as a medal form, a belt form, and a seal form.

In the present system, in a case where there is a manager observing the life of the user or a coach coaching an exercise of the user, a location (output destination) to which information is output from the sensor device 1C, or information based on the information is presented may be set to the information terminal 2, and the information terminal 2 may be operated by the manager or the coach. A location (output destination) to which information is present may be the family of the user of the electronic apparatus 1, a manager of a cabin, and a companion of the mountain climbing party. Information may be presented (output) to any user via the network 3 and the server 4.

The user may mount only the electronic apparatus 1 without using the sensor device 1C. In this case, the electronic apparatus 1 may directly present information output from at least one sensor built into the electronic apparatus 1, to the user without using communication.

### 1-3. Method of Living Body Monitoring Method

### 1-3-1. Overview

The system (an example of a living body monitoring system) according to the present embodiment includes an output unit which outputs status information (an example of information regarding a load applied on the user's body) indicating a physical condition of the user on the basis of a height increase speed (an example of information regarding an altitude change of a point where the user is located) of the user and an oxygen saturation decrease speed (an example of information regarding a change in a blood oxygen amount of the user; hereinafter, referred to as an “SpO₂ decrease speed”) of the user. The status information is information indicating a physical condition of the user as any one of three stages such as “danger”, “slight danger”, and “normality”. The status information is based on both of a height increase speed and an SpO₂ decrease speed of the user, and is thus considered to accurately reflect (predict) a physical condition of the user in a case where an exercise is continuously performed in this state. Therefore, the user may use the status information as a reference for determining whether or not the content of the exercise performed by the user is to be reconsidered. Herein, a case is exemplified in which the status information is expressed in three stages such as “danger”, “slight danger”, and “normality”, but the number of stages may be more than “three”, and may be “two”.

In the system of the present embodiment, elements which can operate as the output unit (output interface) are the display section 170C and the sound output section 180C of the sensor device 1C, the display section 170 and the sound output section 180 of the electronic apparatus 1, the display section 25 and the sound output section 26 of the information terminal 2, and the like. However, herein, as an example, a description will be made of a case where the electronic apparatus 1 of the system can operate alone. In this case, elements which can operate as the output unit are the display section 170, the sound output section 180, and the like of the electronic apparatus 1. In this case, a program stored in the storage section 130 of the electronic apparatus 1 is an example of a living body monitoring program according to the invention, a storage medium into which the program is written is an example of a storage medium according to the invention, a method of a process performed by the processing section 120 is an example of a living body monitoring method according to the invention, and the display section 170 is an example of a display device according to the invention.

### 1-3-2. Table

### 1-3-2-1. Overview of Table

In the system of the present embodiment, the electronic apparatus 1 includes the storage section 130 which stores, for example, a table (an example of information regarding a correspondence relationship) regarding a correspondence relationship between a combination of a height increase speed and an SpO₂ decrease speed of the user, and status information as illustrated in any of FIGS. 7 to 14.

The processing section 120 of the electronic apparatus 1 calculates, for example, a height increase speed of the user at the present time on the basis of a change amount per unit time of an output (an output related to an altitude) from the GPS sensor 110 at the present time.

The processing section 120 of the electronic apparatus 1 calculates, for example, an SpO₂ decrease speed of the user at the present time on the basis of a change amount per unit time of an output (an output related to SpO₂) from the GPS sensor 110 at the present time.

The processing section 120 refers to a table (for example, any of tables illustrated in FIGS. 7 to 14) in the storage section 130 according to a combination of the calculated height increase speed and the SpO₂ decrease speed so as to specify status information correlated with the combination as status information which is to be output to the user.

Therefore, according to the table (for example, any of tables illustrated in FIGS. 7 to 14) in the storage section 130, the processing section 120 can reduce a calculation amount in the processing section 120 when acquiring status information on the basis of the height increase speed of the user at the present time and the SpO₂ decrease speed of the user at the present time.

### 1-3-2-2. Level Based Table

For example, the storage section 130 stores a table for each level of physical ability of the user as illustrated in FIGS. 7 to 9.

On the other hand, the user inputs a level (distinction among a normal person, an intermediate, and an athlete) of physical ability of the user to the electronic apparatus 1 via the operation section 150. The level input by the user is written to the storage section 130 as one of user data.

When status information of the user is acquired, the processing section 120 determines a level of the physical ability of the user on the basis of the user data of the user, reads a table prepared for the level from the storage section 130, and uses the table to acquire status information.

Therefore, the processing section 120 can acquire status information suitable for each of various users having different levels of physical abilities.

### 1-3-2-2-1. Table for Normal People

A table for normal people (FIG. 7) is a table storing status information for each combination of a height increase speed and an SpO₂ decrease speed of a normal user. In this table, as a height increase speed becomes higher, status information indicating a higher degree of danger is correlated therewith, and, as an SpO₂ decrease speed becomes higher, status information indicating a higher degree of danger is correlated therewith.

In other words, status information “danger” indicating the highest degree of danger is correlated with a combination of the highest height increase speed and the highest SpO₂ decrease speed, and status information “normality” indicating the lowest degree of danger is correlated with a combination of the lowest height increase speed and the lowest SpO₂ decrease speed.

Therefore, the probability that the status information “danger” regarding a high degree of danger may be output becomes higher when a normal user moves up at a high speed along a steep slope than when the normal user moves up at a low speed along a gentle slope. The probability that the status information “danger” regarding a high degree of danger may be output becomes higher when an SpO₂ decrease speed of a normal user is high than when the SpO₂ decrease speed of the normal user is low.

A division number in a table, a division pattern of a table (classification of status information), the number of pieces of status information, and the like are not limited to those illustrated in FIG. 7.

This table is set so that status information which is output to a normal user is appropriate at all times. Thus, this table is preferably designed on the basis of statistical information regarding normal users.

### 1-3-2-2-2. Table for Intermediates

A table for intermediates (FIG. 8) is a table storing status information for each combination of a height increase speed and an SpO₂ decrease speed of an intermediate user. In this table, as a height increase speed becomes higher, status information indicating a higher degree of danger is correlated therewith, and, as an SpO₂ decrease speed becomes higher, status information indicating a higher degree of danger is correlated therewith.

In other words, status information “danger” indicating the highest degree of danger is correlated with a combination of the highest height increase speed and the highest SpO₂ decrease speed, and status information “normality” indicating the lowest degree of danger is correlated with a combination of the lowest height increase speed and the lowest SpO₂ decrease speed.

Therefore, the probability that the status information “danger” regarding a high degree of danger may be output becomes higher when an intermediate user moves up at a high speed along a steep slope than when the intermediate user moves up at a low speed along a gentle slope. The probability that the status information “danger” regarding a high degree of danger may be output becomes higher when an SpO₂ decrease speed of an intermediate user is high than when the SpO₂ decrease speed of the intermediate user is low.

A division number in a table, a division pattern of a table (classification of status information), the number of pieces of status information, and the like are not limited to those illustrated in FIG. 8.

This table is set so that status information which is output to an intermediate user is appropriate at all times. Thus, this table is preferably designed on the basis of statistical information regarding intermediate users.

The intermediate user indicates a user having higher skill in exercise (herein, mountain climbing) than that of a normal user and having lower skill in exercise (herein, mountain climbing) than that of an athlete user. The “skill” mentioned here indicates body's adaptability (ability of incorporating oxygen into blood) for changes in an altitude and oxygen saturation is high.

### 1-3-2-2-3. Table for Athletes

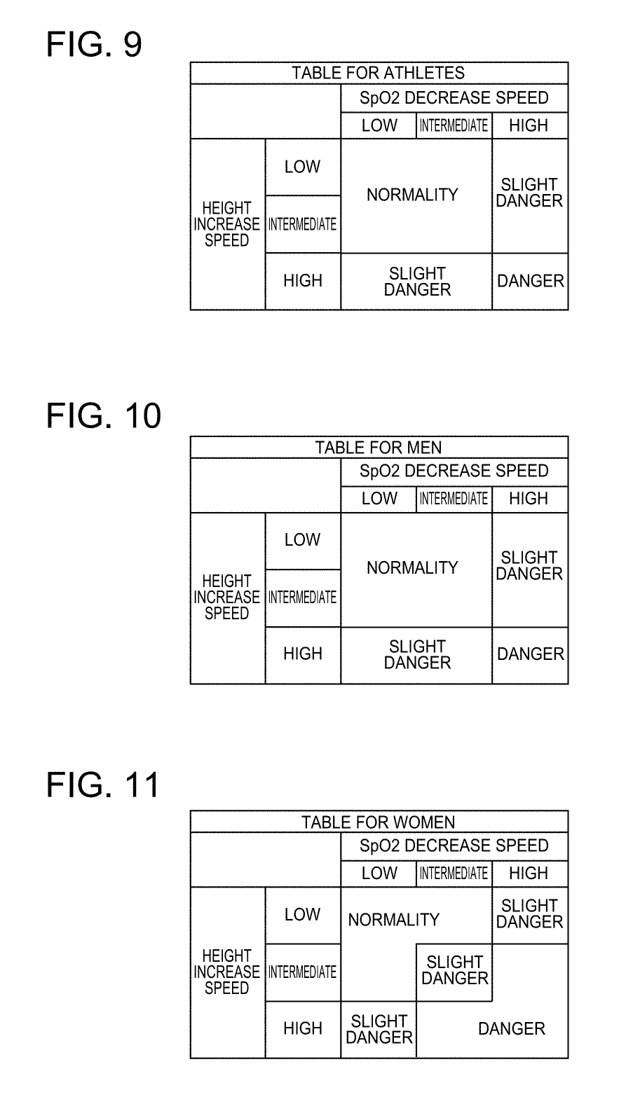

A table for athletes (FIG. 9) is a table storing status information for each combination of a height increase speed and an SpO₂ decrease speed of an athlete user. In this table, as a height increase speed becomes higher, status information indicating a higher degree of danger is correlated therewith, and, as an SpO₂ decrease speed becomes higher, status information indicating a higher degree of danger is correlated therewith.

In other words, status information “danger” indicating the highest degree of danger is correlated with a combination of the highest height increase speed and the highest SpO₂ decrease speed, and status information “normality” indicating the lowest degree of danger is correlated with a combination of the lowest height increase speed and the lowest SpO₂ decrease speed.

Therefore, the probability that the status information “danger” regarding a high degree of danger may be output becomes higher when an athlete user moves up at a high speed along a steep slope than when the athlete user moves up at a low speed along a gentle slope. The probability that the status information “danger” regarding a high degree of danger may be output becomes higher when an SpO₂ decrease speed of an athlete user is high than when the SpO₂ decrease speed of the athlete user is low.

A division number in a table, a division pattern of a table (classification of status information), the number of pieces of status information, and the like are not limited to those illustrated in FIG. 9.

This table is set so that status information which is output to an athlete user is appropriate at all times. Thus, this table is preferably designed on the basis of statistical information regarding athlete users.

The athlete user indicates a user having higher skill in exercise (herein, mountain climbing) than that of an intermediate user. The “skill” mentioned here indicates body's adaptability (ability of incorporating oxygen into blood) for changes in an altitude and oxygen saturation is high.

### 1-3-2-3. Tables for Men and Women

For example, the storage section 130 stores a table for each sex of users as illustrated in FIGS. 10 and 11.

On the other hand, the user inputs the sex (distinction between a man and a woman) of the user to the electronic apparatus 1 via the operation section 150. The sex input by the user is written to the storage section 130 as one of user data.

When status information of the user is acquired, the processing section 120 determines the sex of the user on the basis of the user data of the user, reads a table prepared for the sex from the storage section 130, and uses the table to acquire status information.

Therefore, the processing section 120 can acquire status information suitable for each of various users having different sexes.

### 1-3-2-3-1. Table for Men

A table for men (FIG. 10) is a table storing status information for each combination of a height increase speed and an SpO₂ decrease speed of a male user. In this table, as a height increase speed becomes higher, status information indicating a higher degree of danger is correlated therewith, and, as an SpO₂ decrease speed becomes higher, status information indicating a higher degree of danger is correlated therewith.

In other words, status information “danger” indicating the highest degree of danger is correlated with a combination of the highest height increase speed and the highest SpO₂ decrease speed, and status information “normality” indicating the lowest degree of danger is correlated with a combination of the lowest height increase speed and the lowest SpO₂ decrease speed.

Therefore, the probability that the status information “danger” regarding a high degree of danger may be output becomes higher when a male user moves up at a high speed along a steep slope than when the male user moves up at a low speed along a gentle slope. The probability that the status information “danger” regarding a high degree of danger may be output becomes higher when an SpO₂ decrease speed of a male user is high than when the SpO₂ decrease speed of the male user is low.

A division number in a table, a division pattern of a table (classification of status information), the number of pieces of status information, and the like are not limited to those illustrated in FIG. 10.

This table is set so that status information which is output to a male user is appropriate at all times. Thus, this table is preferably designed on the basis of statistical information regarding male users.

### 1-3-2-3-2. Table for Women

A table for women (FIG. 11) is a table storing status information for each combination of a height increase speed and an SpO₂ decrease speed of a female user. In this table, as a height increase speed becomes higher, status information indicating a higher degree of danger is correlated therewith, and, as an SpO₂ decrease speed becomes higher, status information indicating a higher degree of danger is correlated therewith.

In other words, status information “danger” indicating the highest degree of danger is correlated with a combination of the highest height increase speed and the highest SpO₂ decrease speed, and status information “normality” indicating the lowest degree of danger is correlated with a combination of the lowest height increase speed and the lowest SpO₂ decrease speed.

Therefore, the probability that the status information “danger” regarding a high degree of danger may be output becomes higher when a female user moves up at a high speed along a steep slope than when the female user moves up at a low speed along a gentle slope. The probability that the status information “danger” regarding a high degree of danger may be output becomes higher when an SpO₂ decrease speed of a female user is high than when the SpO₂ decrease speed of the female user is low.

A division number in a table, a division pattern of a table (classification of status information), the number of pieces of status information, and the like are not limited to those illustrated in FIG. 11.

This table is set so that status information which is output to a female user is appropriate at all times. Thus, this table is preferably designed on the basis of statistical information regarding female users.

### 1-3-2-4. Age Based Tables

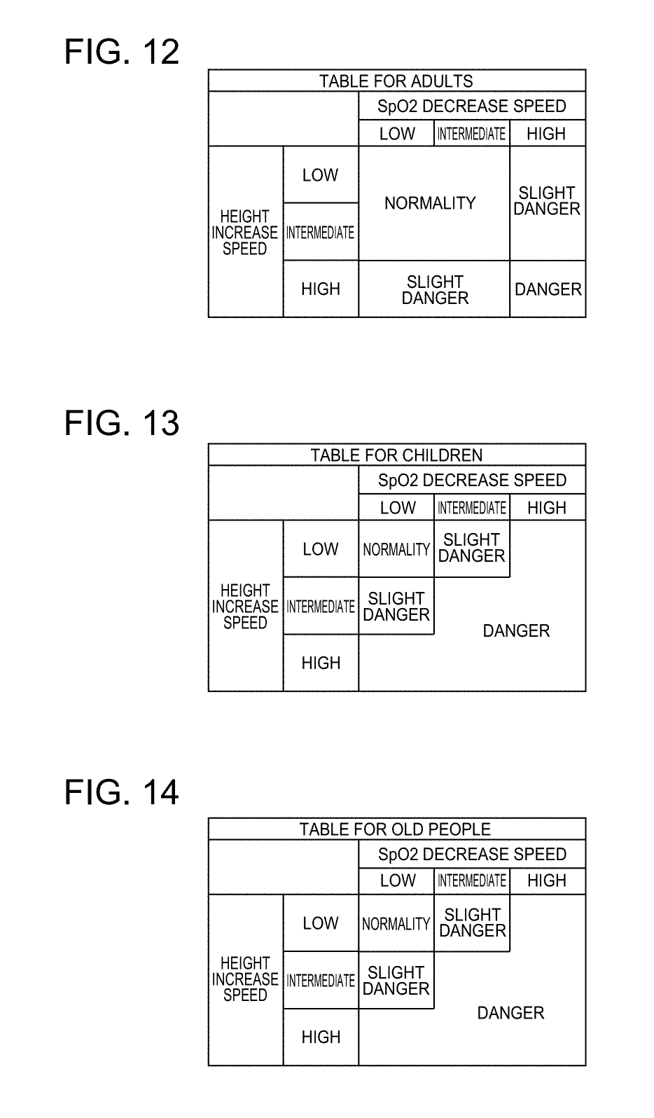

For example, the storage section 130 may store tables corresponding to ages (that is, for each age group or each age) of users as illustrated in FIGS. 12 to 14.

On the other hand, the user inputs the age (age group) of the user to the electronic apparatus 1 via the operation section 150. The age (age group) input by the user is written to the storage section 130 as one of user data.

When status information of the user is acquired, the processing section 120 determines the age (age group) of the user on the basis of the user data of the user, reads a table prepared for the age (age group) from the storage section 130, and uses the table to acquire status information.

Therefore, the processing section 120 can acquire status information suitable for each of various users in different ages.

### 1-3-2-4-1. Table for Adults

A table for adults (FIG. 12) is a table storing status information for each combination of a height increase speed and an SpO₂ decrease speed of an adult user. In this table, as a height increase speed becomes higher, status information indicating a higher degree of danger is correlated therewith, and, as an SpO₂ decrease speed becomes higher, status information indicating a higher degree of danger is correlated therewith.

In other words, status information “danger” indicating the highest degree of danger is correlated with a combination of the highest height increase speed and the highest SpO₂ decrease speed, and status information “normality” indicating the lowest degree of danger is correlated with a combination of the lowest height increase speed and the lowest SpO₂ decrease speed.

Therefore, the probability that the status information “danger” regarding a high degree of danger may be output becomes higher when an adult user moves up at a high speed along a steep slope than when the adult user moves up at a low speed along a gentle slope. The probability that the status information “danger” regarding a high degree of danger may be output becomes higher when an SpO₂ decrease speed of an adult user is high than when the SpO₂ decrease speed of the adult user is low.

A division number in a table, a division pattern of a table (classification of status information), the number of pieces of status information, and the like are not limited to those illustrated in FIG. 12.

This table is set so that status information which is output to an adult user is appropriate at all times. Thus, this table is preferably designed on the basis of statistical information regarding adult users.

The adult user indicates a user in an age group from, for example, sixteen to sixty years old (the “adult” mentioned here does not indicate a legal adult but indicates a physical adult). An age group of adult users is not limited thereto.

### 1-3-2-4-2. Table for Children

A table for children (FIG. 13) is a table storing status information for each combination of a height increase speed and an SpO₂ decrease speed of a child user. In this table, as a height increase speed becomes higher, status information indicating a higher degree of danger is correlated therewith, and, as an SpO₂ decrease speed becomes higher, status information indicating a higher degree of danger is correlated therewith.

In other words, status information “danger” indicating the highest degree of danger is correlated with a combination of the highest height increase speed and the highest SpO₂ decrease speed, and status information “normality” indicating the lowest degree of danger is correlated with a combination of the lowest height increase speed and the lowest SpO₂ decrease speed.

Therefore, the probability that the status information “danger” regarding a high degree of danger may be output becomes higher when a child user moves up at a high speed along a steep slope than when the child user moves up at a low speed along a gentle slope. The probability that the status information “danger” regarding a high degree of danger may be output becomes higher when an SpO₂ decrease speed of a child user is high than when the SpO₂ decrease speed of the child user is low.

A division number in a table, a division pattern of a table (classification of status information), the number of pieces of status information, and the like are not limited to those illustrated in FIG. 13.

This table is set so that status information which is output to a child user is appropriate at all times. Thus, this table is preferably designed on the basis of statistical information regarding children users.

The child user indicates a user in an age lower than that of an adult user (the “child” mentioned here does not indicate a legal child but indicates a physical child). An age group of children users is not limited thereto.

### 1-3-2-4-3. Table for Old People

A table for old people (FIG. 14) is a table storing status information for each combination of a height increase speed and an SpO₂ decrease speed of an old user. In this table, as a height increase speed becomes higher, status information indicating a higher degree of danger is correlated therewith, and, as an SpO₂ decrease speed becomes higher, status information indicating a higher degree of danger is correlated therewith.

In other words, status information “danger” indicating the highest degree of danger is correlated with a combination of the highest height increase speed and the highest SpO₂ decrease speed, and status information “normality” indicating the lowest degree of danger is correlated with a combination of the lowest height increase speed and the lowest SpO₂ decrease speed.

Therefore, the probability that the status information “danger” regarding a high degree of danger may be output becomes higher when an old user moves up at a high speed along a steep slope than when the old user moves up at a low speed along a gentle slope. The probability that the status information “danger” regarding a high degree of danger may be output becomes higher when an SpO₂ decrease speed of an old user is high than when the SpO₂ decrease speed of the old user is low.

A division number in a table, a division pattern of a table (classification of status information), the number of pieces of status information, and the like are not limited to those illustrated in FIG. 14.

This table is set so that status information which is output to an old user is appropriate at all times. Thus, this table is preferably designed on the basis of statistical information regarding old users.

The old user indicates a user in an age higher than that of an adult user. An age group of old users is not limited thereto.

### 1-3-2-5. Various Tables

Herein, a description has been made of three kinds of tables such as the level based table, the sex based table, and the age based table as tables stored in the storage section 130, but a level and sex based table, a level and age based table, or a sex and age based table may be stored in the storage section 130. A level, sex and age based table may be stored in the storage section 130. In other words, in the present system, users may be subdivided into groups by attributes, and a table may be prepared for each subdivided group.

### 1-3-2-6. Customizing of Table

For example, at least one (existing table) of the above-described tables (FIGS. 7 to 14) may be set (adjusted) according to a relationship between a height increase speed and an SpO₂ decrease speed of a user during normal times. In this case, the processing section 120 may cope with a variation between individual users. The normal times indicate a past period in which a user performed an exercise while maintaining a normal physical condition (health state).

Herein, a case is assumed in which various pieces of measured data related to an exercise (herein, mountain climbing) performed by a user in the past are stored in the storage section 130 of the electronic apparatus 1, and the measured data is managed for each date. This is because a user performing mountain climbing continuously uses the electronic apparatus 1 according to the present embodiment so as to be able to accumulate measured data during mountain climbing performed a plurality of times (past history). In the present system, the table is set (adjusted) on the basis of the past history, and thus appropriate status information can be generated by a user.

For example, the user inputs information (for example, the date) which is required to specify measured data during normal times to the electronic apparatus 1 via the operation section 150. The date is the date on which the user could safely and comfortably perform mountain climbing.

The processing section 120 specifies measured data during normal times among pieces of measured data in the storage section 130 on the basis of the information, and calculates a combination of a height increase speed and an SpO₂ decrease speed at each time point by referring to an altitude and SpO₂ at each time point included in the measured data during normal times.

The processing section 120 plots the calculated combinations in a height increase speed-decrease speed graph, and calculates a curve (or a straight line) fitted to plotted points. The processing section 120 adjusts a division pattern (classification of status information) of the existing table according to deviation between the curve and a standard curve (or a standard straight line), and stores the adjusted table in the storage section 130 as a table (customized table) dedicated to the user. Thereafter, when status information of the user is calculated, the customized table is used.

### 1-3-3. Overview of Various Display Screens

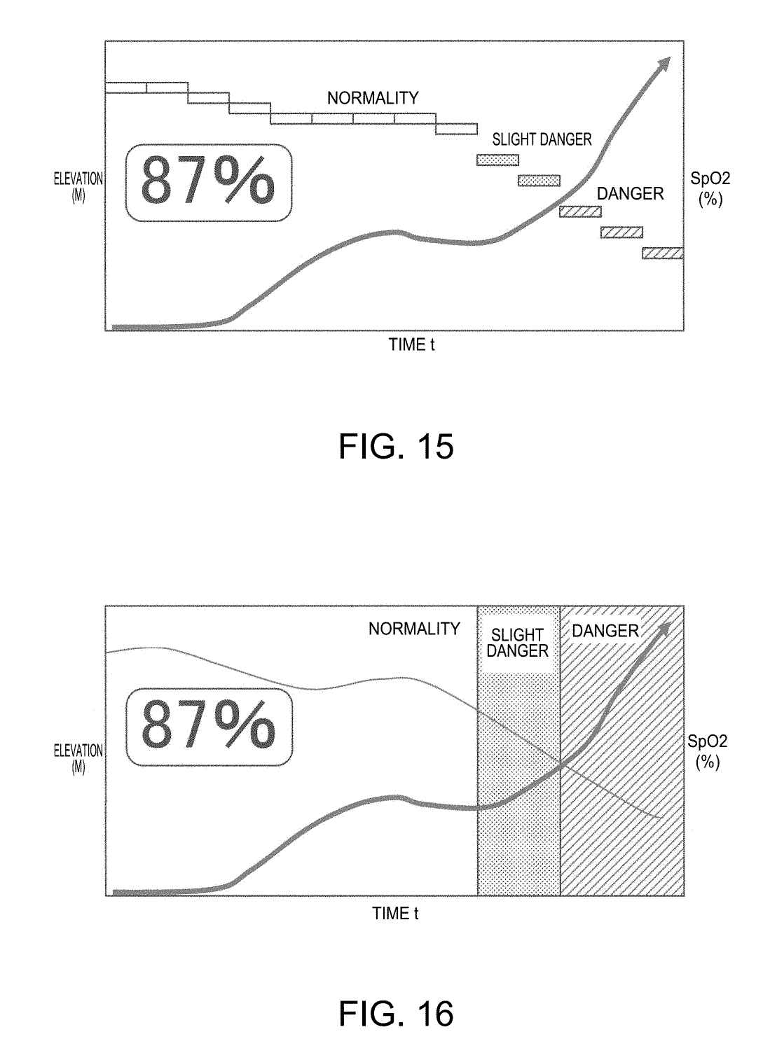

The display section 170 of the electronic apparatus 1 displays status information (distinction among “normality”, “slight danger”, and “danger”) indicating a physical condition of a user as illustrated in FIGS. 15 to 19 on the basis of a height increase speed of the user and an SpO₂ decrease speed of the user along with SpO₂ (a text image such as “87%”) of the user. A display timing is, for example, each time point during a period in which the user performs an exercise (mountain climbing) or a timing desired by the user during a period in which the user performs an exercise (mountain climbing). Therefore, the user can check SpO₂ of the user along with status information of the user's body.

The display section 170 of the electronic apparatus 1 may output transition in status information (distinction among “normality”, “slight danger”, and “danger”) as illustrated in FIGS. 15 to 19 along with transition in SpO₂. In FIGS. 15 to 19, a curve descending toward the right or a block train arranged to descend toward the right indicates transition in SpO₂. In this case, the user can check transition in SpO₂ along with transition in status information. For example, the display section 170 of the electronic apparatus 1 displays transition in SpO₂ in a graph as illustrated in FIGS. 15 to 19. In this case, the user can visually check transition in SpO₂ along with transition in status information.

The display section 170 of the electronic apparatus 1 may represent transition in status information with at least one of, for example, a color and a pattern of a graph as illustrated in FIGS. 15 to 19. In this case, the user can intuitively recognize transition in status information on the basis of at least one of the color and the pattern.

The display section 170 of the electronic apparatus 1 may display, for example, at least one of a graph of transition in an altitude and a graph of transition in atmospheric pressure along with a graph of transition in SpO₂ as illustrated in FIGS. 15 to 19. Therefore, the user can check at least one of the graph of transition in an altitude and the graph of transition in atmospheric pressure along the graph of transition in SpO₂.

### 1-3-3-1. Display Screen in FIG. 15

For example, the display section 170 of the electronic apparatus 1 displays a graph as illustrated in FIG. 15.

A transverse axis of the graph in FIG. 15 is a time axis, and a longitudinal axis thereof is an altitude axis or an SpO₂ axis.

In FIG. 15, transition in an altitude is indicated by a curve, transition in SpO₂ is indicated by a block train, and a block at each time point is added with a color or a pattern corresponding to status information at the time point. The color includes a hue, saturation, or luminance (grayscale), or a combination thereof.

Therefore, the user can recognize a value of SpO₂ at a time point on the basis of a position of a block at each time point, and can recognize status information (distinction among “normality”, “slight danger”, and “danger”) at the time point on the basis of a color or a pattern of the block at each time point.

The text image “87%” disposed in the graph in FIG. 15 indicates a value of SpO₂ of the user at the present time point. In FIG. 15, text images such as “normality”, “slight danger”, and “danger” are displayed, but the distinction among “normality”, “slight danger”, and “danger” is represented by colors or patterns of the blocks, and thus the display of the text images may be omitted. Some of the information in FIG. 15 may not be displayed according to the user's request.

### 1-3-3-2. Display Screen in FIG. 16

For example, the display section 170 of the electronic apparatus 1 displays a graph as illustrated in FIG. 16.

A transverse axis of the graph in FIG. 16 is a time axis, and a longitudinal axis thereof is an altitude axis or an SpO₂ axis.

In FIG. 16, transition in an altitude is indicated by a curve, transition in SpO₂ is also indicated by a curve, and respective regions (time periods) of the graph partitioned in the time direction are added with colors or patterns corresponding to status information (distinction among “normality”, “slight danger”, and “danger”) at the time periods. The color includes a hue, saturation, or luminance (grayscale), or a combination thereof.

Therefore, the user can recognize a value of SpO₂ at a time point on the basis of the curve of SpO₂, and can recognize status information (distinction among “normality”, “slight danger”, and “danger”) at the time period on the basis of a color or a pattern of each region (time period).

The text image “87%” disposed in the graph in FIG. 16 indicates a value of SpO₂ of the user at the present time point. In FIG. 16, text images such as “normality”, “slight danger”, and “danger” are displayed, but the distinction among “normality”, “slight danger”, and “danger” is represented by colors or patterns of the regions (time periods), and thus the display of the text images may be omitted. Some of the information in FIG. 16 may not be displayed according to a user's request.

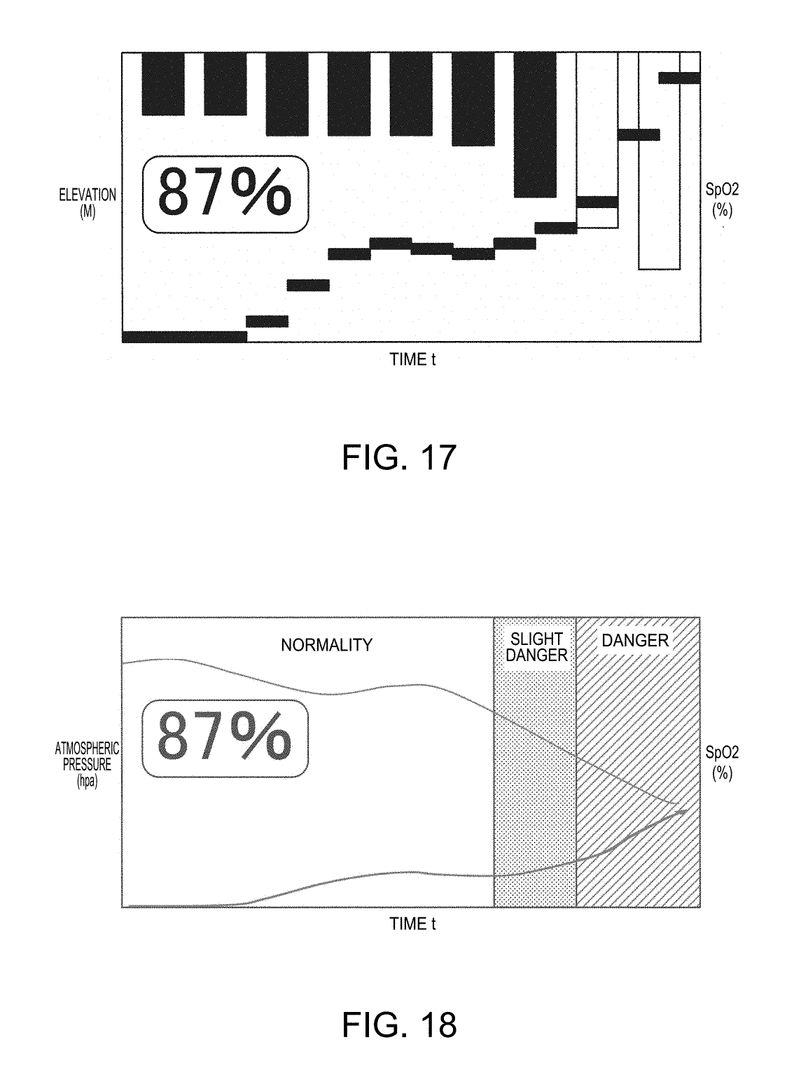

### 1-3-3-3. Display Screen in FIG. 17

For example, the display section 170 of the electronic apparatus 1 displays a graph as illustrated in FIG. 17.

A transverse axis of the graph in FIG. 17 is a time axis, and a longitudinal axis thereof is an altitude axis or an SpO₂ axis.

In FIG. 17, transition in an altitude is indicated by a block train, transition in SpO₂ is indicated by bar graphs, and a bar graph at each time point is added with a color or a pattern corresponding to status information (distinction among “normality”, “slight danger”, and “danger”) at the time point. The color includes a hue, saturation, or luminance (grayscale), or a combination thereof. Each bar graph illustrated in FIG. 17 is represented by a block extending from an upper end side toward a lower end side with respect to the orientation of the drawing surface, and a length of each block indicates a reduction amount from 100% of SpO₂.

Therefore, the user can recognize a value of SpO₂ at a time point on the basis of a length of the bar graph of SpO₂, and can recognize status information (distinction among “normality”, “slight danger”, and “danger”) at the time point on the basis of a color or a pattern of the bar graph at each time point. In the example illustrated in FIG. 17, the block of the bar graph corresponding to “danger” is displayed white, and the block of the bar graph corresponding to “slight danger” or “normality” is displayed black.

The text image “87%” disposed in the graph in FIG. 17 indicates a value of SpO₂ of the user at the present time point. Some of the information in FIG. 17 may not be displayed according to the user's request.

### 1-3-3-4. Display Screen in FIG. 18

For example, the display section 170 of the electronic apparatus 1 displays a graph as illustrated in FIG. 18.

A transverse axis of the graph in FIG. 18 is a time axis, and a longitudinal axis thereof is an atmospheric pressure axis or an SpO₂ axis.

In FIG. 18, transition in atmospheric pressure is indicated by a curve, transition in SpO₂ is also indicated by a curve, and respective regions (time periods) partitioned in the time direction are added with colors or patterns corresponding to status information (distinction among “normality”, “slight danger”, and “danger”) at the time periods. The color includes a hue, saturation, or luminance (grayscale), or a combination thereof.

Therefore, the user can recognize a value of SpO₂ at a time point on the basis of the curve of SpO₂, and can recognize status information (distinction among “normality”, “slight danger”, and “danger”) at the time period on the basis of a color or a pattern of each region (time period).

The text image “87%” disposed in the graph in FIG. 18 indicates a value of SpO₂ of the user at the present time point. In FIG. 18, text images such as “normality”, “slight danger”, and “danger” are displayed, but the distinction among “normality”, “slight danger”, and “danger” is represented by colors or patterns of the regions (time periods), and thus the display of the text images may be omitted. Some of the information in FIG. 18 may not be displayed according to a user's request.

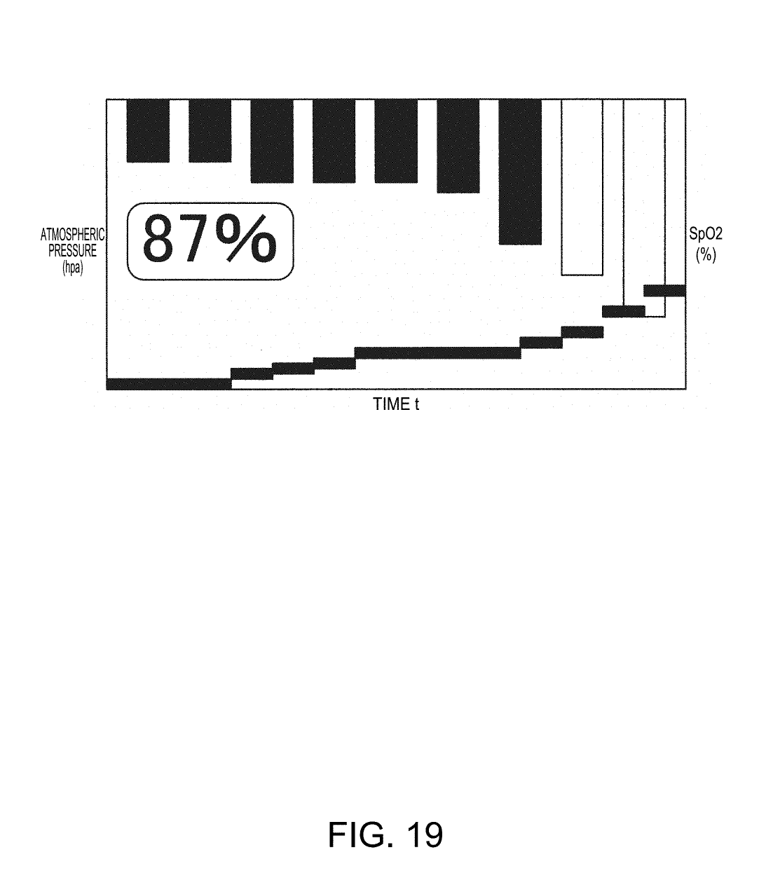

### 1-3-3-5. Display Screen in FIG. 19

For example, the display section 170 of the electronic apparatus 1 displays a graph as illustrated in FIG. 19.

A transverse axis of the graph in FIG. 19 is a time axis, and a longitudinal axis thereof is an atmospheric pressure axis or an SpO₂ axis.

In FIG. 19, transition in atmospheric pressure is indicated by a block train, transition in SpO₂ is indicated by bar graphs, and a bar graph at each time point is added with a color or a pattern corresponding to status information (distinction among “normality”, “slight danger”, and “danger”) at the time point. The color includes a hue, saturation, or luminance (grayscale), or a combination thereof. Each bar graph illustrated in FIG. 19 is represented by a block extending from an upper end side toward a lower end side with respect to the orientation of the drawing surface, and a length of each block indicates a reduction amount from 100% of SpO₂.

Therefore, the user can recognize a value of SpO₂ at a time point on the basis of a length of the bar graph of SpO₂, and can recognize status information (distinction among “normality”, “slight danger”, and “danger”) at the time point on the basis of a color or a pattern of the bar graph at each time point. In the example illustrated in FIG. 19, the block of the bar graph corresponding to “danger” is displayed white, and the block of the bar graph corresponding to “slight danger” or “normality” is displayed black.

The text image “87%” disposed in the graph in FIG. 19 indicates a value of SpO₂ of the user at the present time point. Some of the information in FIG. 19 may not be displayed according to the user's request.

### 1-3-4. Flow

Next, a description will be made of a flow in a case where the electronic apparatus 1 mounted on a certain user issues a warning corresponding to status information (distinction among “normality”, “slight danger”, and “danger”) to the user during an exercise (herein, mountain climbing) by using an image or a sound.

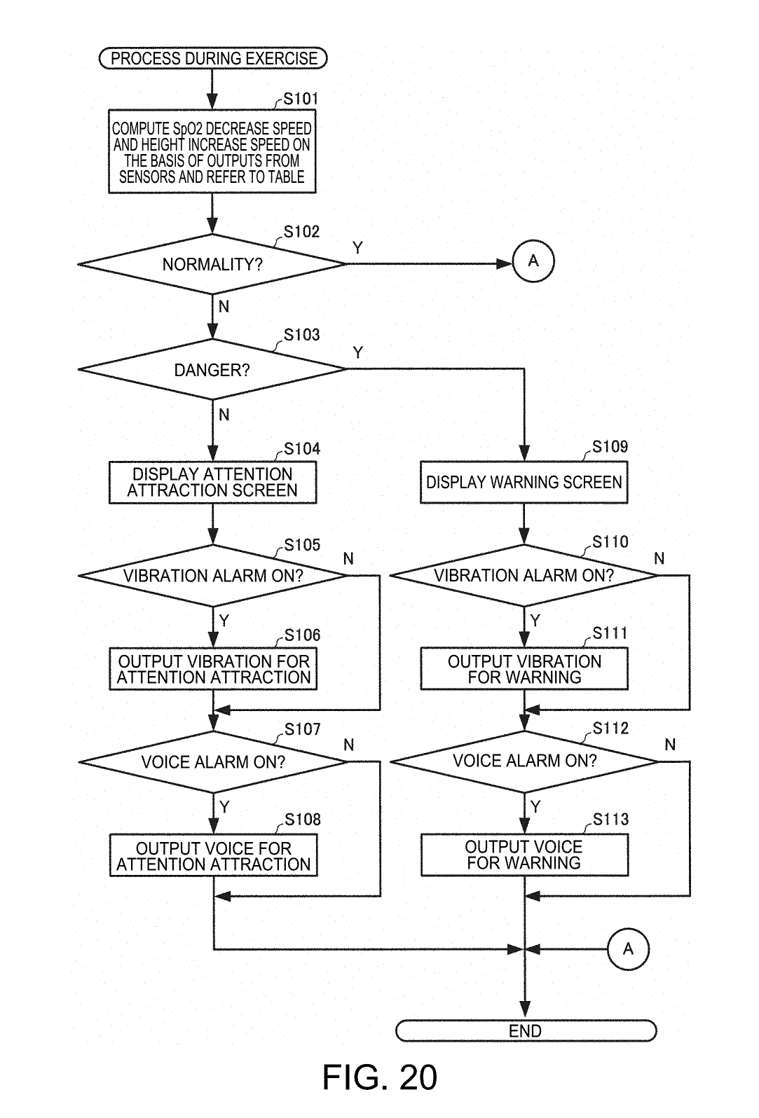

FIG. 20 illustrates an example of a flowchart regarding a process performed by the processing section 120 of the electronic apparatus 1. The flowchart is repeatedly executed by the processing section 120 of the electronic apparatus 1 during a period in which the user performs mountain climbing. A frequency of execution is a predetermined frequency such as 1/60 seconds.

If the flow is started, the processing section 120 computes an SpO₂ decrease speed and a height increase speed on the basis of data output from sensors (for example, the SpO₂ sensor 117, the GPS sensor 110, and the atmospheric pressure sensor 112), and refers to a table stored in the storage section 130 according to a combination of the computed SpO₂ decrease speed and height increase speed, so as to specify status information correlated with the combination (S101).

In step S101, the processing section 120 refers to change amounts per unit time of the outputs from the sensors (for example, the SpO₂ sensor 117, the GPS sensor 110, and the atmospheric pressure sensor 112) in order to compute the SpO₂ decrease speed and the height increase speed. For this, the processing section 120 may use positive values (or the history) of data output from the sensors (for example, the SpO₂ sensor 117, the GPS sensor 110, and the atmospheric pressure sensor 112). The table referred to by the processing section 120 in step S101 is a table conforming to user data (a level, the sex, the age, or the like of the user) of the user or a table (adjusted table) customized for the user in advance.

Next, the processing section 120 determines whether or not the specified status information corresponds to “normality” (S102), finishes the flow in a case where the status information corresponds to “normality” (S102Y), and proceeds to the next determination step (S103) if otherwise (S102N). In a case where the status information corresponds to “normality”, any one of the above-described graphs is continuously displayed (normal display), and a warning using vibration or a voice is not issued.

Next, the processing section 120 determines whether or not the status information corresponds to “danger” (S103), proceeds to a warning process (S109) in a case where the status information corresponds to “danger” (S103Y), and proceeds to an attention attraction process (S104) if otherwise (S103N).

Next, the processing section 120 displays an attention attraction screen on the display section 170 (S104). The attention attraction screen is a screen in which, for example, an emphasis process for attention attraction has been performed on any one of the above-described graphs. The emphasis process may be performed through emphasis of a color of the entire screen, may be performed through a temporal change (blinking display or the like) of the entire screen, and may also be performed by adding an image (a text image, a mark, or an icon) for attention attraction to a part of the screen. The color includes a hue, saturation, or luminance (grayscale), or a combination thereof.

Next, the processing section 120 determines whether or not vibration output (vibration alarm) is permitted (ON) in advance by the user (S105). It is assumed that information indicating that a vibration alarm is permitted is stored in the storage section 130. The processing section 120 proceeds to the next process (S106) if the processing section 120 refers to the storage section 130 and recognizes that the vibration alarm is permitted (S105Y), and skips the next process (S106) if otherwise (S105N).

Next, the processing section 120 causes the sound output section 180 to output vibration for attention attraction (S106).

Next, the processing section 120 determines whether or not voice output (voice alarm) is permitted (ON) in advance by the user (S107). It is assumed that information indicating that a voice alarm is permitted is stored in the storage section 130. The processing section 120 proceeds to the next process (S108) if the processing section 120 refers to the storage section 130 and recognizes that the voice alarm is permitted (S107Y), and skips the next process (S108) if otherwise (S107N), and then finishes the flow.

Next, the processing section 120 causes the sound output section 180 to output a voice for attention attraction (S108), and finishes the flow.

On the other hand, in a case where the status information corresponds to “danger” in step S103 (S103Y), the processing section 120 displays a warning screen on the display section 170 (step S109). The warning screen is a screen in which an emphasis process for warning has been performed on any one of the above-described graphs.

Next, the processing section 120 determines whether or not vibration output (vibration alarm) is permitted (ON) in advance by the user (S110). It is assumed that information indicating that a vibration alarm is permitted is stored in the storage section 130. The processing section 120 proceeds to the next process (S111) if the processing section 120 refers to the storage section 130 and recognizes that the vibration alarm is permitted (S110Y), and skips the next process (S111) if otherwise (S110N).

Next, the processing section 120 causes the sound output section 180 to output vibration for warning (S111).

Next, the processing section 120 determines whether or not voice output (voice alarm) is permitted (ON) in advance by the user (S112). It is assumed that information indicating that a voice alarm is permitted is stored in the storage section 130. The processing section 120 proceeds to the next process (S113) if the processing section 120 refers to the storage section 130 and recognizes that the voice alarm is permitted (S112Y), and skips the next process (S113) if otherwise (S112N), and then finishes the flow.

Next, the processing section 120 causes the sound output section 180 to output a voice for warning (S113), and finishes the flow.

In the above-described flow, the order of execution of some of the steps may be replaced with each other in an allowable range. Some of the steps in the flow may be omitted.

### 1-3-5. Advice

In the electronic apparatus 1 of the present embodiment, status information output to a user may include advice to the user. For example, status information of “normality” may include advice related to pace retention (load retention) such as the content that “please continuously perform the exercise at the current pace”, status information of “slight danger” may include advice related to pace-down (load reduction) such as the content that “please reduce your pace”, and status information of “danger” may include advice related to exercise stoppage (load exclusion) such as the content that “please immediately stop the exercise”. In this case, the user can check status information of the user as advice to the user.

In the electronic apparatus 1 of the present embodiment, advice may include at least one of advice related to the necessity of rest and advice related to the necessity of doctor's examination. For example, the status information of “danger” may include advice such as the content that “please take a doctor's examination”. Therefore, it is possible to recommend doctor's examination to the user at right timing corresponding to status information (distinction among “normality”, “slight danger”, and “danger”) of the user's body.

### 1-3-6. Navigation

In the electronic apparatus 1 of the present embodiment, status information may include at least one of information regarding a point where a rest is possible and information regarding a point where a doctor's examination is possible. In this case, it is possible to notify the user of at least one of information regarding a point where a rest is possible and information regarding a point where a doctor's examination is possible at right timing corresponding to status information (distinction among “normality”, “slight danger”, and “danger”) of the user's body.

For example, in a case where it is determined that status information corresponds to “danger”, the processing section 120 of the electronic apparatus 1 may cause the electronic apparatus 1 to develop a well-known navigation function so as to search for medical institutions such as hospitals or clinics in an area in which the present point of the user is included and to guide (navigate) the user to a medical institution closest to the present point. Navigating may be performed by using an image, a sound, or light, or a combination thereof.

### 1-3-7. Variation of Notification Aspects

In the electronic apparatus 1 of the present embodiment, status information may be information which can be recognized by the user through at least one of an auditory sense, a visual sense, and a tactile sense. In this case, the status information can be recognized by the user through any one of the user's auditory sense, visual sense, and tactile sense.

In the electronic apparatus 1 of the present embodiment, the status information may be displayed by using at least one of a color, text, a symbol, a scale, and a pattern which can be recognized through a visual sense by the user. In this case, the status information can be recognized by the user by using at least one of a color, text, a symbol, a scale, and a pattern.

### 1-3-8. Output Destination Variation

The system of the present embodiment may include the portable electronic apparatus 1 which can be carried by the user, and another portable electronic apparatus (for example, the electronic apparatus (not illustrated) has the same configuration as that of the electronic apparatus 1) which can be another user who is different from the user. At least one of output destinations of status information of the user may be the portable electronic apparatus of another user. In this case, since another user can understand the status information of the user, for example, in a case where the user loses consciousness, another user can be aware thereof so as to perform immediate handling. “Another user” mentioned here may be an accompanying user included in the same mountain climbing party as that of the user, and may be another user present near the user.

### 2. OPERATIONS AND EFFECTS OF EMBODIMENT

As described above, the electronic apparatus 1 according to the present embodiment outputs status information (distinction among “normality”, “slight danger”, and “danger”) indicating a physical condition of the user on the basis of a height increase speed of a user and an SpO₂ decrease speed of the user. The status information is based on both of a height increase speed and an SpO₂ decrease speed of the user, and is thus considered to accurately reflect (predict) a physical condition of the user in a case where an exercise is continuously performed in this state. Therefore, the user may use the status information as a reference for determining whether or not the content of the exercise performed by the user is to be reconsidered.

Since the electronic apparatus 1 according to the present embodiment measures heartbeat, body movement and position information, stores the information in association with time information, analyzes and displays the information, performs communication with other apparatuses, and causes a blood oxygen amount to be included in measurement, analysis and display items, for example, it is possible to recognize the process of a user reaching altitude sickness during mountain climbing, or the illness occurrence by using indexes or through warning, and thus to prevent the occurrence of serious illness.

The electronic apparatus 1 according to the present embodiment includes the atmospheric pressure sensor and the GPS sensor so as to be able to associate an altitude with blood oxygen concentration data and thus to analyze change amounts in a height increase speed and a blood oxygen amount during mountain climbing, and can thus provide prediction or a warning of altitude sickness to a user.

Activities at high places such as mountain climbing may cause altitude sickness symptoms (altitude disorder) due to hypoxic, and, specifically, symptoms such as headache, nausea, or sleep disorder may be caused. Severe symptoms such as cerebral edema and pulmonary edema may be caused depending on cases, but it is possible to avoid these risks in advance.

### 3. MODIFICATION EXAMPLES

### 3-1. Content of Table

At least any of the above-described tables and advice may be created by a manager of the system, a manufacturer of the electronic apparatus 1, a supervisor of the system, or the like on the basis of medical information.

In the above-described tables, status information (advice) of a user is set to three stages such as “normality” (pace retention), “slight danger” (pace-down), and “danger” (stoppage), but the number of stages may be increased. For example, status information (advice) may be set to five stages such as (i) “load deficiency” (pace-up), (ii) “normality” (pace retention), (iii) “slight danger” (pace-down), (iv) “danger” (rest), and (v) “extreme danger” (mountain descending or a request for rescue).

3-2. Combination with Other Indexes

In the above-described system, there may be a configuration in which a value of SpO₂ of a user is monitored, and, in a case where the value is smaller than a predetermined threshold value, a warning (strong warning) is issued to the user so that an exercise is immediately stopped or a request for rescue is made, regardless of the above-described status information.

In the system, the above-described table may be prepared for each piece of atmospheric pressure (oxygen partial pressure), and the table may be used according to atmospheric pressure.

In the system, the above-described table may be prepared for each piece of weather, and the table may be used according to weather.

In the system, the above-described table may be prepared for each pulse rate, and the table may be used according to a pulse rate of a user.

In the system, the above-described table may be prepared for each body temperature, and the table may be used according to a body temperature of a user.

History information (past measured data) of an SpO₂ decrease speed and a height increase speed may be used to set a table. More various processes can be performed on the basis of other measured data. For example, the history information includes environmental information. In this case, the storage section may store, as the history information, information in which the environmental information is correlated with an SpO₂ decrease speed and a height increase speed, and the processing section 120 may generate notification information on the basis of the SpO₂ decrease speed and the height increase speed correlated with the environmental information.

For example, a value of SpO₂ and an altitude when the value is acquired are stored in correlation with each other as history information. In the above-described way, a user can store characteristics of the target user that a value of SpO₂ is Y (%) at an altitude of about X (m). Thus, a physical condition of the user can be estimated through comparison between a value Y′ (%) of SpO₂ when the user reaches a point of the altitude X (m) in the latest mountain climbing and the past record value Y of the target user stored as the history information.

For example, if Y′≥Y, SpO₂ which is equivalent to or more than the past record is maintained in the present mountain climbing, and thus it is considered that there is no problem in a physical condition. On the other hand, if Y′<Y, it may be determined that there is a tendency that the reduction extent of SpO₂ is more rapid in the present mountain climbing than in the past mountain climbing, and a physical condition is bad. It may be estimated to what extent a physical condition is bad according to a difference between Y′ and Y. If there is the tendency that a physical condition is bad, the processing section 120 generates notification information for prompting pace-down, rest, or repose.

Information correlated with SpO₂ may be environmental information in addition to an altitude, and may be information other than environmental information. For example, history information may be information in which SpO₂, environmental information, and biological information of a user are correlated with each other. In this case, the storage section 130 stores information in which the environmental information and the biological information of the user are correlated with SpO₂ as history information, and the processing section 120 may generate notification information on the basis of SpO₂ correlated with the environmental information and the biological information of the user.

For example, a value of SpO₂, and an altitude and a pulse rate when the value is acquired are stored in correlation with each other as history information. In the above-described way, a user can store characteristics of the target user that a value of SpO₂ is Y (%) and a pulse rate is Z (beats/minute) at an altitude of about X (m). The processing section 120 can determine whether or not a physical condition of the user is bad by using not only comparison between the above Y′ and Y but also Z corresponding to the history of a pulse rate. The processing section 120 may change the content of notification information or a notification timing on the basis of a determination result.

For example, an evaluation function E (X, Y, Z, Y′, Z′) having X, Y, and Z which are history information, a value Y′ of the present SpO₂ and the present pulse rate Z′ as parameters (variables) may be set, and a physical condition of the user may be estimated on the basis of the magnitude of a value of the evaluation function. A process in the processing section 120 based on history information may be variously modified.

Whether an actual physical condition is good or bad may depend on subjectivity of a user to some extent . Therefore, in a case where a user feels altitude sickness symptoms such as headache during mountain climbing, the user may perform input on the electronic apparatus 1. The storage section 130 of the electronic apparatus 1 stores the fact that the user feels a bad physical condition at an input timing, and performs a process by using the stored information in the subsequent generation of notification information.

For example, it is assumed that, in a case where an altitude is X, a value of SpO₂ is Y, and a pulse rate is Z, the storage section stores that there is input from a user. In this case, the values of the set (X, Y, and Z) are not standard values to the target user, and are values indicating a dangerous state to the extent of feeling a bad physical condition. Therefore, in a case where the same tendency is observed at the subsequent timing, it is determined that there is a tendency for a physical condition of the user to be bad, and notification information for prompting the user to rest as soon as possible is generated.

As described above, a case where the user is aware of a bad physical condition corresponds to a case where a low oxygen state lasts for a long period of time to some extent. Also from the viewpoint of preventing severe symptoms, the electronic apparatus 1 preferably notifies the user of alert information or advice information before reaching dangerous values such as stored (X, Y, and Z). For example, simply, the processing section 120 may perform a process of generating and outputting alert information at a timing before an altitude reaches X (m) in the latest mountain climbing on the basis of history information that the user feels a bad physical condition around the altitude of X (m). Similarly, the processing section 120 may generate and output alert information before SpO₂ is reduced to about Y or before a pulse rate is changed to about Z.

For example, a value of SpO₂, an altitude, a pulse rate, and information (0=no input, and 1=input) indicating whether or not there is input from a user, acquired at a predetermined timing ti (where i=0, 1, . . . ) are stored. For example, values such as SpO₂=95.5, an altitude=100, a pulse rate=85, and user input=0 are acquired at a timing t0, and are stored in correlation with each other in a first row.

Timings (acquisition rates or calculation rates) at which respective pieces of information such as SpO₂, an altitude, and a pulse rate are acquired cannot be said to match each other. Therefore, history information stored in the storage section is not necessarily limited to information in which SpO₂, environmental information, and biological information are correlated with each other. For example, there may be a case where a pulse rate is correlated with a predetermined value of SpO₂, but an altitude is not correlated therewith. Correlated data is not limited to pieces of data whose acquisition timings match each other, and data whose acquisition timings are close to each other may be correlated. For example, in a case where there are SpO₂ acquired at a time point T1 and an altitude acquired at a time point T2, if a condition such as |T1−T2|≤δ (where δ is a predetermined threshold value) is satisfied, the two pieces of data may be correlated with each other so as to be stored as history information.

In the present system, a calculation amount is reduced by using the table when status information is generated, but a computation formula may be used instead of using the table. The computation formula is, for example, a formula for calculating an index indicating the degree of danger on the basis of a combination of an SpO₂ decrease speed and a height increase speed. The processing section 120 may calculate an index according to the computation formula, and may generate status information according to a range in which the index is included.

### 3-3. Upload of Measured Data

In the system of the present embodiment, the processing section 120 of the electronic apparatus 1 preserves various pieces of data generated during the above-described measurement in the storage section 130 in a predetermined format as measured data along with date and time data indicating the date and time of the measurement. One or a plurality of pieces of measured data stored in the storage section 130 are transmitted to the information terminal 2 so as to be stored in the storage section 24 of the information terminal 2 as necessary. The measured data stored in the storage section 24 of the information terminal 2 is uploaded to the server 4 as necessary. The measured data uploaded to the server 4 is stored in the storage section 44 of the server 4. The measured data at various dates and times of various users stored in the storage section 44 of the server 4 are managed for each user for each date and time by the processing section 41 of the server 4. Communication between the electronic apparatus 1 and the information terminal 2 is performed via the communication section 190 of the electronic apparatus 1 and the communication section 22 of the information terminal 2, and communication between the information terminal 2 and the server 4 is performed via the communication section 27 of the information terminal 2, the network 3, and the communication section 42 of the server 4.

### 3-4. Variation of System Configuration

If the system of the present embodiment includes a portable apparatus having a sensor which can be mounted on a user's body, any one of the electronic apparatus 1, the sensor device 1C, the information terminal 2, and the server 4 may be omitted.

Sharing of functions in the system is not limited to the above description. For example, some or all of the functions of the sensor device 1C may be installed in the electronic apparatus 1, and may be installed in the information terminal 2. Some or all of the functions of the electronic apparatus 1 may be installed in the sensor device 1C, and may be installed in the information terminal 2. Some or all of the functions of the information terminal 2 may be installed in the sensor device 1C, and may be installed in the electronic apparatus 1. Some or all of the functions of the information terminal 2 may be installed in the server 4, and some of the functions of the sever 4 may be installed in the information terminal 2. For example, measured data generated by the sensors may be sequentially uploaded to the server 4 from the electronic apparatus 1, and necessary information may be sequentially calculated by the server 4 so as to be sequentially transmitted to the electronic apparatus 1.

### 3-5. Variation of Sensor

The sensor device 1C or the electronic apparatus 1 of the embodiment may use at least one of the following various sensors as a sensor. In other words, the various sensors are, for example, an acceleration sensor, a GPS (GNSS) sensor, an angular velocity sensor, a speed sensor, a heartbeat sensor (a chest belt or the like), a pulse sensor (a sensor performing measurement at locations other than the heart), a pedometer, a pressure sensor, an altitude sensor, a temperature sensor (an atmospheric temperature sensor or a body temperature sensor), a geomagnetic sensor, a weight meter (which is used as an external device of the system), an ultraviolet sensor, a perspiration sensor, a blood pressure sensor, an arterial blood oxygen concentration (SpO₂) sensor, a lactic acid sensor, a blood sugar level sensor, and a wind speed sensor. A sensor which is not used for measurement may not be mounted in the apparatus in the embodiment.

### 3-6. Notification Aspects

At least one of the electronic apparatus 1, the sensor device 1C, or the information terminal 2 may perform a notification of information for a user through image display, may perform a notification not only through image display but also through sound output or by using vibration, light, or a color (light emission from an LED or a display color of a display), and may perform a notification through a combination of at least two of image display, sound output, vibration, light, and a color.

For example, a value of SpO₂ normally measured is displayed, or, in a case where status information of “danger” is obtained, an alert is output through a combination of at least two of image display, sound output, vibration, light, and a color, and thus it is possible to contribute to prevention of altitude sickness symptoms.

For example, the system notifies a user of status information through graph display, but may notify the user of at least some status information (including advice) through sound output, vibration, light, a color, image display, or a combination thereof.

### 3-7. Customizing

At least some of the notification content (including a notification period, a notification item, a notification aspect, a collecting method, a notification order, and the like) for a user in the electronic apparatus 1, the sensor device 1C, and the information terminal 2 of the embodiment may be set in advance by the user (customizable).

### 3-8. Forms of Apparatus

The electronic apparatus 1 may be configured as portable information terminals of various types, such as a wrist type electronic apparatus, an earphone type electronic apparatus, a ring type electronic apparatus, a pendant type electronic apparatus, an electronic apparatus attached to a sport appliance and used, a smart phone, a head mounted display (HMD), and a head up display (HUD). However, in a case where measured data obtained by the SpO₂ sensor 117 is essential, a modification of the electronic apparatus 1 is performed within the scope of without obstructing measurement in the SpO₂ sensor 117.

### 3-9. Optional Functions

Other functions may be installed in at least one of the sensor device 1C, the electronic apparatus 1, and the information terminal 2. Other functions may be, for example, well-known smart phone functions. The smart phone functions include, for example, a call function, a mail incoming notification function, a call incoming notification function, a communication function, and a camera function.

### 3-10. Positioning System

In the embodiment, as a satellite positioning system, a global positioning system (GPS) is used, but a global navigation satellite system (GNSS) may be used. For example, one or two or more of satellite positioning systems such as a European Geostationary-Satellite Navigation Overlay Service (EGNOS), a quasi zenith satellite system (QZSS), a global navigation satellite system (GLONASS), GALILEO, a BeiDou navigation satellite system (BeiDou) may be used. As at least one of the satellite positioning systems, a satellite-based augmentation system (SBAS) such as European geostationary-satellite navigation overlay service (EGNOS) or a wide area augmentation system (WAAS) may be used.

### 3-11. Example of HMD

Hereinafter, a description will be made of an example of a head mounted display with reference to FIG. 21.

A head mounted display 100 includes an image display section 20 (display section) which enables a user to visually recognize a virtual image in a state of being mounted on the head of the user, and a control device 10 which controls the image display section 20. The control device 10 also functions as a controller used for the user to operate the head mounted display 100. The image display section 20 is a mounting body which is mounted on the head of the user, and has a spectacle type frame 2′ (main body) in the present embodiment.

As the control device 10, the information terminal 2 illustrated in FIG. 1 may be used. In this case, the operation section 23 of the information terminal 2 functions as a determination key 11, a display changing key 13, a track pad 14, a luminance changing key 15, a direction key 16, a menu key 17, and the like. FIG. 16 illustrates an example in which the image display section 20 and the control device 10 are connected to via a connection unit 40 (a main body cord 48′, a right cord 42′, a left cord 44′, and a cable 48), but the image display section 20 and the control device 10 may be connected to each other through wireless communication.

The frame 2′ has a right holding unit 21′ and a left holding unit 23′ . The right holding unit 21′ is a member which is provided so as to extend over a position corresponding to the temporal region of the user from an end part ER which is the other end of a right optical image display unit 26′ when the user wears the image display section 20. Similarly, the left holding unit 23′ is a member which is provided so as to extend over a position corresponding to the temporal region of the user from an end part EL which is the other end of a left optical image display unit 28′ when the user wears the image display section 20. The right holding unit 21′ comes into contact with the right ear or the vicinity thereof on the head of the user, and the left holding unit 21′ comes into contact with the left ear or the vicinity thereof so that the right holding unit 21′ and the left holding unit 23′ hold the image display section 20 on the head of the user. The right holding unit 21′ and the left holding unit 23′ hold the image display section 20 on the head of the user in the same manner as temples of spectacles.

In the present embodiment, the spectacle type frame 2′ will be described an example of the main body. A shape of the main body is not limited to a spectacle shape, and may be any shape as long as the main body is mounted on and fixed to the head of the user, and is more preferably a shape which causes the main body to be hung in front of both the eyes of the user. For example, in addition to the spectacle shape described here, a shape of the main body may be a snow goggle shape covering the upper part of the face of the user, and may be a shape which is disposed in front of each of the right and left eyes of the user, such as binoculars.

The frame 2′ is provided with a right display driving unit 22′, a left display driving unit 24′, the right optical image display unit 26′, the left optical image display unit 28′, and a microphone 63. The right display driving unit 22′ and the left display driving unit 24′ are disposed on a side opposing the head of the user when the user wears the image display section 20. The right optical image display unit 26′ and the left optical image display unit 28′ are respectively located in front of the right eye and the left eye of the user when the user wears the image display section 20. One end of the right optical image display unit 26′ and one end of the left optical image display unit 28′ are connected to each other at the position corresponding to the glabella of the user when the user wears the image display section 20.

### 4. OTHERS

The invention is not limited to the above-described embodiment, and may be variously modified within the scope of the spirit of the invention.

The above-described embodiment and modification examples are only examples, and the invention is not limited thereto. For example, the embodiment and the respective modification examples may be combined with each other as appropriate.

The invention includes substantially the same configuration (for example, a configuration in which functions, methods, and results are the same, or a configuration in which objects and effects are the same) as the configuration described in the embodiment. The invention includes a configuration in which an inessential part of the configuration described in the embodiment is replaced with another part. The invention includes a configuration which achieves the same operation and effect or a configuration capable of achieving the same object as in the configuration described in the embodiment. The invention includes a configuration in which a well-known technique is added to the configuration described in the embodiment.

The entire disclosure of Japanese Patent Application No. 2016-223387, filed Nov. 16, 2016, is expressly incorporated by reference herein.

What is claimed is:

1. A living body monitoring system comprising:
a sensor device that includes a processing section generating information regarding a load applied on a user's body on the basis of information regarding a change in an altitude of a point where the user is located and information regarding a change in a blood oxygen amount of the user; and
an output unit that outputs the information regarding the load.

2. The living body monitoring system according to claim 1,
wherein the sensor device further includes
a storage section that stores information regarding a correspondence relationship between a combination of the information regarding a change in the altitude and the information regarding a change in the blood oxygen amount, and the information regarding the load.

3. The living body monitoring system according to claim 2,
wherein the storage section stores the information regarding the correspondence relationship for each level of physical ability of the user.

4. The living body monitoring system according to claim 2,
wherein the storage section stores the information regarding the correspondence relationship for each sex of the user.

5. The living body monitoring system according to claim 2,
wherein the storage section stores the information regarding the correspondence relationship according to the age of the user.

6. The living body monitoring system according to claim 2,
wherein the information regarding the correspondence relationship is set according to a relationship between the information regarding a change in the altitude and the information regarding a change in the blood oxygen amount during normal times.

7. The living body monitoring system according to claim 1,
wherein the output unit outputs transition in the load along with transition in the blood oxygen amount.

8. The living body monitoring system according to claim 7,
wherein the output unit displays the transition in the blood oxygen amount in a graph.

9. The living body monitoring system according to claim 1,
wherein the information regarding the load includes advice to the user.

10. The living body monitoring system according to claim 9,
wherein the advice includes at least one of advice related to the necessity of rest and advice related to the necessity of doctor's examination.

11. The living body monitoring system according to claim 9,
wherein the information regarding the load includes at least one of information regarding a point where a rest is possible and information regarding a point where a doctor's examination is possible.

12. The living body monitoring system according to claim 9,
wherein the information regarding the load is information which can be recognized by the user through at least one of an auditory sense, a visual sense, and a tactile sense.

13. The living body monitoring system according to claim 9,
wherein the output unit includes a portable electronic apparatus which can be carried by the user, and a portable electronic apparatus which can be carried by another user who is different from the user, and
wherein at least one of output destinations of the information regarding the load is the portable electronic apparatus of another user.

14. A living body monitoring method comprising:
outputting information regarding a load applied on a user's body on the basis of information regarding a change in an altitude of a point where the user is located and information regarding a change in a blood oxygen amount of the user.

15. A portable electronic apparatus comprising:
a positioning data generation section that generates positioning data;
a sensor that measures a blood oxygen amount of a user; and
at least one processing section that generates information regarding a change in an altitude based on the positioning data and information regarding a change in the blood oxygen amount of the user based on a measurement result of the blood oxygen amount of the user,
wherein the processing section outputs information regarding a load applied on the user's body on the basis of the information regarding a change in the altitude and the information regarding a change in the blood oxygen amount, to a display.

16. A display method comprising:
displaying a load applied on a user's body on the basis of information regarding a change in an altitude of a point where the user is located and information regarding a change in a blood oxygen amount of the user, along with the blood oxygen amount.

17. The display method according to claim 16,
wherein transition in the load is displayed along with transition in the blood oxygen amount.

18. The display method according to claim 17,
wherein the transition in the blood oxygen amount is displayed in a graph.

19. The display method according to claim 18,
wherein the transition in the load is indicated by at least one of a color and a pattern of the graph.

20. The display method according to claim 19,
wherein at least one of a graph of transition in the altitude and a graph of transition in atmospheric pressure is displayed along with the graph of transition in the blood oxygen amount.
